SEQUENCE LISTING (22 sequences)

SEQ ID NO: 1 - DNA, 23 bp, synthetic construct
  source 1..23: mol_type = other DNA
gttggtccattgatgtttagtct

SEQ ID NO: 2 - DNA, 23 bp, synthetic construct
  source 1..23: mol_type = other DNA
ttggaaaacagggcttgttcaaa

SEQ ID NO: 3 - DNA, 23 bp, synthetic construct
  source 1..23: mol_type = other DNA
tagcacgcttcctccatgctcat

SEQ ID NO: 4 - DNA, 23 bp, synthetic construct
  source 1..23: mol_type = other DNA
tatttatccatgtagaatttcat

SEQ ID NO: 5 - DNA, 23 bp, synthetic construct
  source 1..23: mol_type = other DNA
atgatgttgttgatcattgaatt

SEQ ID NO: 6 - DNA, 23 bp, synthetic construct
  source 1..23: mol_type = other DNA
tctgcaggagtcatggtcaaacc

SEQ ID NO: 7 - DNA, 23 bp, synthetic construct
  source 1..23: mol_type = other DNA
caaagttaggaatggccaacgct

SEQ ID NO: 8 - DNA, 23 bp, synthetic construct
  source 1..23: mol_type = other DNA
cacatacttggaaaacagggctt

SEQ ID NO: 9 - DNA, 23 bp, synthetic construct
  source 1..23: mol_type = other DNA
aaccactctgaagttgcccacgt

SEQ ID NO: 10 - DNA, 23 bp, synthetic construct
  source 1..23: mol_type = other DNA
ggtgacagatgattgtagcccaa

SEQ ID NO: 11 - RNA, 23 nt, synthetic construct
  source 1..23: mol_type = other RNA
gttggtccattgatgtttagtct

SEQ ID NO: 12 - RNA, 23 nt, synthetic construct
  source 1..23: mol_type = other RNA
ttggaaaacagggcttgttcaaa

SEQ ID NO: 13 - RNA, 23 nt, synthetic construct
  source 1..23: mol_type = other RNA
tagcacgcttcctccatgctcat

SEQ ID NO: 14 - RNA, 23 nt, synthetic construct
  source 1..23: mol_type = other RNA
tatttatccatgtagaatttcat

SEQ ID NO: 15 - RNA, 23 nt, synthetic construct
  source 1..23: mol_type = other RNA
atgatgttgttgatcattgaatt

SEQ ID NO: 16 - RNA, 23 nt, synthetic construct
  source 1..23: mol_type = other RNA
tctgcaggagtcatggtcaaacc

SEQ ID NO: 17 - RNA, 23 nt, synthetic construct
  source 1..23: mol_type = other RNA
caaagttaggaatggccaacgct

SEQ ID NO: 18 - RNA, 23 nt, synthetic construct
  source 1..23: mol_type = other RNA
cacatacttggaaaacagggctt

SEQ ID NO: 19 - RNA, 23 nt, synthetic construct
  source 1..23: mol_type = other RNA
aaccactctgaagttgcccacgt

SEQ ID NO: 20 - RNA, 23 nt, synthetic construct
  source 1..23: mol_type = other RNA
ggtgacagatgattgtagcccaa

SEQ ID NO: 21 - DNA, 23 bp, synthetic construct
  source 1..23: mol_type = other DNA
gtgaacagctcctcgcccttgct

SEQ ID NO: 22 - DNA, 21 bp, synthetic construct
  source 1..21: mol_type = other DNA
gctgaacttgtggccgtttac

(19) United States
(12) Patent Application Publication
(10) Pub. No.: US 2025/0381302 A1
(43) Pub. Date: Dec. 18, 2025

(54) METHODS OF PREVENTING OR TREATING AN RNA VIRAL INFECTION IN A SUBJECT

(71) Applicants: Agency for Science, Technology and Research, Singapore (SG); National University of Singapore, Singapore (SG)

(72) Inventors: Wei Leong Chew, Singapore (SG); Choong Tat Keng, Singapore (SG); Jang Hann Chu, Singapore (SG); Thinesshwary Yogarajah, Singapore (SG)

(21) Appl. No.: 18/838,911

(22) PCT Filed: Feb. 16, 2023
(86) PCT No.: PCT/SG2023/050089

§ 371 Date: Aug. 15, 2024

(30) Foreign Application Priority Data
Feb. 16, 2022 (SG) 10202201473X

(51) Int. Cl. A61K 48/00; A61K 35/76; A61P 31/14

(52) U.S. Cl. CPC A61K 48/0058; A61K 35/76; A61K 48/0083; A61P 31/14

(57) ABSTRACT

The present invention relates generally to the field of molecular biology. In particular, the specification teaches methods of preventing or treating an RNA viral infection in a subject.

### FIELD OF INVENTION

The present invention relates generally to the field of molecular biology. In particular, the specification teaches methods of preventing or treating an RNA viral infection in a subject.

### BACKGROUND

Enterovirus 71 (EV71), Coxsackievirus (CAV16 and CAV6) and Echovirus are non-enveloped, positive single stranded RNA viruses that are highly contagious and spread through bodily fluids. EV71 infection is most common in children younger than 5 years of age, with about 50-80% of children tested seropositive for EV71, and is also observed in adults to a lesser extent. Because of the symptoms associated with the infection, EV71 is also a major contributor to the disease known as Hand Foot and Mouth Disease (HFMD). The infection may occasionally result in severe neurological diseases or death. To date, there is no commercially available vaccine or therapeutics for the prevention or elimination of EV71 infection. Clinical trials are ongoing only for vaccine modalities and not therapeutic modalities. In recent years, EV71 infections have reached record incidence rates in several countries such as but not limited to Singapore, Malaysia and China, and this highlights a pressing need to develop vaccines and therapeutics against the currently incurable infections.

It would be desirable to overcome or ameliorate at least one of the above-described problems, or at least to provide a useful alternative.

### SUMMARY

Disclosed herein is a method of preventing or treating an RNA viral infection in a subject, the method comprising administering at a dose of about 5×10¹¹ to about 5×10¹³ vgs/kg of a recombinant adeno-associated virus (AAV) to the subject, wherein the AAV comprises at least one heterologous nucleic acid sequence encoding a Cas13 nuclease and one or more guide RNAs.

Disclosed herein is a recombinant adeno-associated virus (AAV) for use in treating an RNA viral infection in a subject, wherein about 5×10¹¹ to about 5×10¹³ vgs/kg of the recombinant AAV is to be administered to the subject, wherein the AAV comprises at least one heterologous nucleic acid sequence encoding a Cas13 nuclease and one or more guide RNAs.

Disclosed herein is the use of a recombinant adeno-associated virus (AAV) in the manufacture of a medicament for treating an RNA viral infection in a subject, wherein about 5×10¹¹ to about 5×10¹³ vgs/kg of the recombinant AAV is to be administered to the subject, wherein the AAV comprises at least one heterologous nucleic acid sequence encoding a Cas13 nuclease and one or more guide RNAs.

Disclosed herein is a method of inhibiting an RNA viral nucleic acid in a subject, the method comprising administering about 5×10¹¹ to about 5×10¹³ vgs/kg of a recombinant adeno-associated virus (AAV) to the subject, wherein the AAV comprises at least one heterologous nucleic acid sequence encoding a Cas13 nuclease and one or more guide RNAs.

In one embodiment, the method comprises inhibiting the RNA viral nucleic acid in a skeletal or central nervous system (CNS) cell or tissue of the subject.

### BRIEF DESCRIPTION OF DRAWINGS

Embodiments of the present invention are hereafter described, by way of non-limiting example only, with reference to the accompanying drawings in which:

FIG. 1. Design of new antiviral AAV-CRISPR-CasRx therapeutics for RNA virus targeting. A. Schematics of mechanism of hypothesized CRISPR-CasRx antiviral activity on RNA viruses such as EV-A71. Positive strand RNA viruses such as EV-A71 releases the genome upon entry into the host cell, which serves as a template to make negative strand genomic and subgenomic templates, which in turn are then used to produce more copies of the positive strand viral genome and viral mRNAs. AAV delivered CasRx and guide RNAs expressed in the EV-A71 infected cell can specifically target and cleave both the viral genome and the viral mRNA to disrupt viral replication and viral functions. B. Schematic of design of new AAV genomic cargo plasmid for anti-viral application. Plasmid of pZac2.1-CMV-CasRx-3×HA-PolyA-U6-gRNA. Between the ITR sequences, CMV promoter drives the expression of CasRx with a 3×HA tag and without NLS sequence, followed by a rabbit polyadenylation sequence. This is followed by the U6 promoter which drives the transcription of the inserted guide sequences and guide scaffold followed by another ITR sequence. Guide sequences can be cloned into this backbone using the BbsI restriction enzyme digestion. C. To validate that the packaged viruses are able to transduce mammalian cells and express the CasRx cargo, 10K human immortalized corneal endothelial cells, B4G12, were transduced and plated in a 48 well plate at MOI 100K for 72 h. Anti-HA staining is carried out at 1:200 for 2 h and anti-GFP at 1:1500 for 2 h. Secondary antibodies staining is carried out at 1:1000 for 2 h. Images are taken at exposure 15 ms and gain set at 6400 for bright field and the 488 channel, exposure is set at 800 us and gain at 6400 for the DAPI channel. D. Validation of AAVs with new cargo plasmid bearing CasRx and gRNA. Human muscle immortalized cell line, RD, were transduced with AAV2-GFP at MOI 10K for expression of GFP. The GFP knockdown efficiency of guide1 only, guide2 only and guide1+guide2 are tested by transduction of the GFP-expressing RD cells with AAV2-CasRx bearing the GFP-targeting guides at MOI 100K (D) or MOI 1000K (E) for 72 h.

FIG. 2. CRISPR-CasRx gRNAs selection and antiviral targeting strategies. A. Schematic of bioinformatics pipeline for gRNA prediction and scoring, as applied to enterovirus genomes. B. Visualization of scores and gRNAs against 795 enterovirus target genomic sequences. Top: Entropy Score for each nucleotide of aligned viral genomes. Middle: Percentage of Conservation for each nucleotide between aligned viral genomes. Bottom: Frequencies of gRNAs that reside within the best 4th quartile for targeting enteroviruses. C. The selected gRNAs targeting the enterovirus RNA genome. Blue arrows: gRNAs with high scores. Red arrows: gRNAs with medium/low scores. D. Assessment of low scoring guides-Inhibitory activities of EV-A71 replication under single guide single target condition or multiple guide single target condition. In a 96 well plate, 10K RD cells were seeded in each well and transduced with AAVDJ-eGFP with no guides or AAVDJ-CasRx bearing either eGFP guide2 or EV-A71 guide(s) for 72 h and then subjected to EV-A71 infection at MOI of 1 for 12 h, the supernatant is then harvested for virus plaque assay. The control AAV-CasRx uses no guides or eGFP guide2. Comparison between two groups (CasRx-eGFPg2 and CasRx-EV71_3Dguide(s)) was analyzed by Student t test (two-tailed) using the software Prism 9. *p<0.05, **p<0.005. E. Assessment of high scoring guides-Inhibitory activities of EV-A71 replication under single guide single target condition or single/dual guide per target with multiple targets condition. In a 96 well plate, 10K RD cells were seeded in each well and transduced with AAVDJ-eGFP with no guides or AAVDJ-CasRx bearing either eGFP guide2 or EV-A71 guide(s) for 72 h and then subjected to EV71 infection at MOI of 1 for 12 h, the supernatant is then harvested for virus plaque assay. The control AAV-CasRx uses no guides or eGFP guide2. Comparison between two groups (CasRx-eGFPg2 and CasRx-EV71_3Dguide(s)) was analyzed by Student t test (two-tailed) using the software Prism 9. *p<0.05, **p<0.005. F. Assessment on the number of multiplexed CasRx guides on a single target for efficient inhibitory activity on EV-A71 replication. In a 96 well plate, 10K RD cells were seeded in each well and transduced with AAV2-eGFP with no guides or AAV2-CasRx bearing either GFP guide2 or EV-A71 guide(s) at MOI 1000K for 72 h and then subjected to EV71 infection at MOI of 1 for 12 h, the supernatant is then harvested for virus plaque assay. The control AAV2-CasRx uses no guide or eGFP guide2. Comparison between two groups (CasRx-eGFPg2 and CasRx-EV71_3Dguide(s)) was analyzed by Student t test (two-tailed) using the software Prism 9. * p<0.05 G. Percentage of EV-A71 plaques in the presence of different number of multiplexed CasRx guides at MOI 1000K.

FIG. 3. Bio-panning of AAV serotypes to identify AAV serotype capsid that is the most efficient for transduction. A. The human skeletal muscle was proposed to be a target organ that supports EV71 persistent infection and replication. Using the human RD rhabdomyosarcoma cell line (ATCC), an AAV panel bio-panning experiment was performed to determine the most suitable AAV serotype(s) that can efficiently transduce human muscle cells to deliver the CRISPR-CasRx tools for viral RNA targeting. 10K cells were plated into each well of a 96-well plate and transduced with each serotype of AAV at MOI of 100K and the expression of GFP protein was quantitated at 72 h post-transduction. Experiments were performed in duplicates. B. The mouse skeletal muscle is one of the main target organ that supports EV71 persistent infection and replication. Using the mouse C2C12 skeletal muscle cell line (ATCC), an AAV panel bio-panning experiment was performed to determine the most suitable AAV serotype(s) that can efficiently transduce the mouse muscle cells to deliver the CRISPR-CasRx tools for viral RNA targeting in the mouse model. 10K cells were plated into each well of a 96-well plate and transduced with each serotype of AAV at MOI of 10K and the expression of GFP protein was quantitated at 72 h post-transduction. Experiments were performed in duplicates.

FIG. 4. Human innate and adaptive immune response profiling in AAV2-transduced human RD cells. In a 6-well dish, 1 million human skeletal RD cells were seeded and transduced with AAV2 serotype at MOI of 100K and 1000K. RNA of the transduced cells were harvested at 72 hrs post-transduction using the RNeasy universal plus mini kit (Qiagen). cDNA were synthesized and the immune response gene panel was quantitated using the RT2 profiling kit (Qiagen). The result showed that there is minimal antiviral cellular response in the AAV2 transduced RD cells with only two genes (NLRP3 and SLC11A1) significantly upregulated in a dose-dependent manner. NLRP3 and SLC11A1 are both involved in macrophage activation or recruitment but has not been shown to exhibit intracellular antiviral activity.

FIG. 5. In vivo evaluation of antiviral efficacy of AAVDJ-CRISPR-CasRx in a mouse model for hand, foot and mouth disease. A. BALB/c mice were injected intraperitoneally with a dose of 1×10¹¹ vgs or 1×10¹² vgs of AAVDJ-EV71gRNAs per mouse at 2 days old and subsequently injected intraperitoneally with EV-A71 at a dose of 2×10⁷ PFU per mouse at 5 days old. 1×10¹² vgs AAVDJ-GFPgRNA was used as treatment control. The survival of the mice were recorded for 19 days post-infection (dpi). B. The percentage of body weight of each mouse in each treatment group were recorded for 19 dpi. C. The clinical score of each mice were recorded using the mice clinical assessment scoring system (M-CASS) involving observation of five markers: activity, breathing, movement, body weight, and dehydration for up to 19 dpi. D. Virus titers in the hind limbs of mice from different treatment groups, determined using plaque forming assay. E. Virus titers in brain of mice from different treatment groups, determined using plaque forming assay. F. H&E staining of hindlimb and spinal cord of mice. Polymorphonuclear meningitis in the spinal cord (black arrow). Necrosis and focal interstitial mononuclear cell infiltrate in the hind limbs and spinal cord (blue arrow). G. Immuno-histochemistry staining for EV-A71 specific antigen. Presence of viral antigen in the hind limbs and cervical spinal cord anterior horn cells (red arrow).

FIG. 6. In vivo evaluation of antiviral efficacy of AAVDJ-CRISPR-CasRx in a mouse model for hand, foot and mouth disease. A. BALB/c mice were injected intraperitoneally with a dose of 2×10⁷ PFU EV-A71 per mouse at 5 days old. After 2 hours, mice were injected intraperitoneally with a dose of 1×10¹¹ vector genomes (vgs) or 1×10¹² vgs of AAVDJ-EV71gRNAs per mouse. 1×10¹² vgs AAVDJ-GFPgRNA was used as treatment control. The survival of the mice were recorded for 19 days post-infection (dpi). B. The percentage of body weight of each mouse in each treatment group were recorded for 19 dpi. C. The clinical score of each mice were recorded using the mice clinical assessment scoring system (M-CASS) involving observation of five markers: activity, breathing, movement, body weight, and dehydration for up to 19 dpi.

FIG. 7. AAVDJ-CRISPR-CasRx prevents mortality and reduces pathology when applied 6 hrs after EV-A71 infection. A. BALB/c mice were injected intraperitoneally with a dose of 2×10⁷ PFU EV-A71 per mouse at 5 days old. After 6 hours, mice were injected intraperitoneally with a dose of 1×10¹¹ viral genomes (vgs) or 1×10¹² vgs of AAVDJ-EV71gRNAs per mouse. 1×10¹² vgs AAVDJ-GFPgRNA was used as treatment control. B. The survival of the mice was recorded for 19 days post-infection (dpi). Comparison between two groups (CasRx-GFPg2 and CasRx-EV71_3Dguides) was analyzed by Log-rank (Mantel-Cox) test. **p<0.005. C. The body weight of each mouse in each treatment group was recorded for 19 dpi. D. The clinical score of each mouse was recorded using the mice clinical assessment scoring system (M-CASS) for up to 19 dpi. E. Virus titers in the hind limbs of mice as determined using the plaque-forming assay. F. Virus titers in the brains of mice as determined using plaque-forming assay: Kruskal-Wallis test with Dunn's multiple comparisons post hoc test. **p<0.005. G. Immuno-histochemistry staining for EV-A71 specific antigen (red arrow). H. H&E staining of the hind limbs and spinal cords of mice. Polymorphonuclear meningitis in the spinal cord (arrow). Necrosis and focal interstitial mononuclear cell infiltrate in the hind limbs (arrow).

FIG. 8. AAVDJ-CRISPR-CasRx prevents mortality and reduces pathology when applied 24 hrs after EV-A71 infection. A. BALB/c mice were injected intraperitoneally with a dose of 2×10⁷ PFU EV-A71 per mouse at 5 days old. After 24 hours, mice were injected intraperitoneally with a dose of 1×10¹¹ viral genomes (vgs) or 1×10¹² vgs of AAVDJEV71gRNAs per mouse. 1×10¹² vgs AAVDJ-GFPgRNA was used as treatment control. B. The survival of the mice was recorded for 19 days post-infection (dpi). Comparison between two groups (CasRx-GFPg2 and CasRx-EV71_3Dguides) was analyzed by Log-rank (Mantel-Cox) test. *p<0.05. C. The body weight of each mouse in each treatment group was recorded for 19 dpi. D. The clinical score of each mouse was recorded using the mice clinical assessment scoring system (M-CASS). E. Virus titers in the hind limbs of mice from different treatment groups were determined using the plaque-forming assay. F. Virus titers in the brains of mice from different treatment groups were determined using the plaque-forming assay. Comparison between two groups (CasRx-GFPg2 and CasRx-EV71_3Dguides) was analyzed by Kruskal-Wallis test with Dunn's multiple comparisons post hoc test. **p<0.005. G. Immunohistochemistry staining for EV-A71 specific antigen. Presence of viral antigen in the hind limbs (arrow). H. H&E staining of the hind limbs and spinal cord of mice. Polymorphonuclear meningitis in the spinal cord (arrow). Necrosis and focal interstitial mononuclear cell infiltrate in the hind limbs and spinal cord (arrow).

FIG. 9. AAVDJ-CRISPR-CasRx does not induce overt toxicity at the treatment dosages. A. BALB/c mice were injected intraperitoneally with saline, 1×10¹² vgs AAVDJ-GFPgRNA, 1×10¹¹ vgs of AAVDJ-EV71gRNAs, or 1×10¹² vgs of AAVDJEV71gRNAs per 2-day old mouse. Survival was recorded for 19 days post-infection (dpi). B. The body weight of each mouse in each treatment group was recorded for 19 dpi. C. The clinical score of mice was recorded using the mice clinical assessment scoring system (MCASS) involving observation of five markers: activity, breathing, movement, body weight, and dehydration for up to 19 dpi.

FIG. 10. Expression of CasRx-HA in mice transduced with AAVDJCasRx-EV71gRNA. Tissue sections were stained with anti-HA antibodies and counterstained with DAPI. CasRx-HA staining was observed in all groups for both spinal cord and hind limb tissues (top row). Stronger CasRx staining was observed in tissues of mice transduced with 1×10¹² vgs of AAVDJ-GFPgRNA (left column) and 1×10¹² vgs of AAVDJ-EV71gRNA (right column) compared to tissues of mice transduced with 1×10¹¹ vgs AAVDJ-EV71gRNA (centre column).

FIG. 11. Pan-enteroviral inhibition by AAV2-CasRx-EV71_3Dguides. A. Assessment of inhibitory activity of the six-gRNAs pool targeting HFM41 strain on three other EV71 variants (H strain, B5 strain, and C4 strain). 10K RD cells were transduced with AAV2-CasRx at MOI of 10K, 100K, and 1000K bearing either GFP guide2 or EV-A71 guide(s) for 72 h and then subjected to EV-A71 infection at MOI of 1 for 12 h, after which the supernatant is harvested for virus plaque assay. B. The pool of 6 gRNAs designed against the HFM41 strain exhibits antiviral inhibition against three other EV71 variants (H strain, B5 strain, and C4 strain) at an MOI of 1000K. C. Each pool of 6 gRNAs is engineered specifically for the sequences of the H, B5, or C4 strain. D. Each re-engineered pool of 6 gRNAs exhibits antiviral inhibition against their respective H, B5, or C4 strain. In all panels, comparison between two groups (CasRx-GFPg2 and CasRx-EV71_3Dguides) was analyzed by Student t-test (two-tailed). *p<0.05, **p<0.005, ***p<0.001.

### DETAILED DESCRIPTION

The present specification teaches a method of preventing or treating an RNA viral infection in a subject. The method may comprise administering a recombinant adeno-associated virus (AAV) to the subject, wherein the AAV comprises at least one heterologous nucleic acid sequence encoding a Cas13 nuclease and one or more guide RNAs. In one embodiment, the AAV comprises a construct encoding at least one heterologous nucleic acid sequence encoding a Cas13 nuclease and one or more guide RNAs.

In one embodiment, there is provided a method of preventing or treating a RNA virus infection in a subject, the method comprising administering a recombinant adeno-associated virus (AAV) to the subject, wherein the AAV comprises at least one heterologous nucleic acid sequence encoding a Cas13 nuclease and one or more guide RNAs. The method may comprise administering about 5×10¹¹ to about 5×10¹³ vgs/kg of the recombinant adeno-associated virus (AAV) to the subject.

Without being bound by theory, the inventors show for the first time that CRISPR-Cas can eliminate RNA viruses in vivo within the body. The disclosure demonstrates that disease progression can be prevented in an infected mouse model and discloses that disease progression can be treated in an infected mouse model. CRISPR-Cas can also inhibit the RNA viral nucleic acid in a skeletal or central nervous system (CNS) cell or tissue.

Disclosed herein is a method of preventing or treating a RNA virus infection in a subject, the method comprising administering at a dose of about 5×10¹¹ to about 5×10¹³ vector genomes (vgs)/kg of a recombinant adeno-associated virus (AAV) to the subject, wherein the AAV comprises at least one heterologous nucleic acid sequence encoding a Cas13 nuclease and one or more guide RNAs.

The RNA virus infection may be an infection by a single stranded RNA virus. The single stranded RNA virus may be selected from the group consisting of an Enterovirus, a Coxsackie virus and a Parechovirus. The Enterovirus may be Enterovirus 71. The Coxsackie virus may be selected from the group consisting of CAV16 and CAV6. The Parechovirus may be selected from the group consisting of Parechovirus A, Parechovirus B, Parechovirus C, Parechovirus D, Parechovirus E, and Parechovirus F.

### Cas Nuclease

The term “Cas nuclease”, “CRISPR-associated protein”, “Cas protein” or “CRISPR-associated nuclease” refers to a wild type Cas protein, a fragment thereof, or a mutant or variant thereof. The term “Cas mutant” or “Cas variant” refers to a protein or polypeptide derivative of a wild type Cas protein, e.g., a protein having one or more point mutations, insertions, deletions, truncations, a fusion protein, or a combination thereof. In certain embodiments, the Cas mutant or Cas variant substantially retains the nuclease activity of the Cas protein. In certain embodiments, the Cas nuclease is mutated such that one or more nuclease domains are inactive. In some embodiments, the Cas nuclease is mutated so that it lacks some or all of the nuclease activity of its wild-type counterpart. The term “Cas nuclease” also contemplates the use of natural and engineered Cas nucleases.

The term “cleavage” or “cleaving” refers to breaking of the covalent phosphodiester linkage in the ribosylphosphodiester backbone of a polynucleotide. The terms “cleavage” or “cleaving” encompass both single-stranded breaks and double-stranded breaks. A “nuclease cleavage site” or “genomic nuclease cleavage site” is a region of nucleotides that comprise a nuclease cleavage sequence that is recognized by a specific nuclease, which acts to cleave the nucleotide sequence of a polynucleotide.

In one embodiment, the Cas nuclease is a Cas13 nuclease. The Cas nuclease may be a Cas13a, Cas13b, Cas13c or Cas13d nuclease. The Cas13 nuclease may be a Cas13d or CasRx nuclease (which is a Cas 13d from Ruminococcus flavefaciens). The Cas13 nuclease may, for example, show consistent and strong target-specific cleavage effect. It also has a small size for fitting to the AAV delivery system in clinic. The Cas13 nuclease may be an engineered Cas13 nuclease comprising, for example, one or more amino acid substitutions, truncation and/or circular permutation. In one embodiment, the endonuclease function of the Cas13 nuclease is retained in the engineered Cas13 nuclease.

In one embodiment, the Cas nuclease is codon-optimized for expression in a cell, such as in a human cell.

### Guide RNA

In one embodiment, there is provided at least one guide RNA comprises i) a first nucleic acid sequence having at least 70% sequence identity to a nucleic acid sequence encoded by one of the nucleic acid sequences set forth in SEQ ID NO: 1-10, or ii) a first nucleic acid sequence having at least 70% sequence identity to one of the nucleic acid sequences set forth in SEQ ID NO: 11-20.

The method as defined herein may comprise administering 1, 2, 3, 4, 5, 6 or more different guide RNAs to a subject. The method may comprises administering 6 different guide RNAs, wherein each of the guide RNAs comprise i) a first nucleic acid sequence having at least 70% sequence identity to a nucleic acid sequence encoded by one of the nucleic acid sequences set forth in SEQ ID NO: 1-6 or ii) a first nucleic acid sequence having at least 70% sequence identity to one of the nucleic acid sequences set forth in SEQ ID NO: 11-16.

As used herein, the term “guide RNA” or “guide RNA” refers to a RNA which is specific for the target nucleic acid and can form a complex with Cas protein (such as Cas13) and bring Cas protein to the target nucleic acid. The guide RNA may comprise or consist of a spacer sequence (i.e. the first nucleic acid sequence) that is specific to a target nucleic acid and a direct repeat sequence (i.e. the second nucleic acid sequence) that facilitates binding to a Cas protein (such as a Cas13 protein).

The terms “nucleic acid” and “polynucleotide’, used interchangeably herein, refer to polymeric forms of nucleotides of any length, either ribonucleotides or deoxyribonucleotides. Thus, these terms include, but are not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. These terms further include, but are not limited to, mRNA or cDNA that comprise intronic sequences. The backbone of the polynucleotide can comprise sugars and phosphate groups (as may typically be found in RNA or DNA), or modified or substituted sugar or phosphate groups. Alternatively, the backbone of the polynucleotide can comprise a polymer of synthetic subunits such as phosphoramidites and thus can be an oligodeoxynucleoside phosphoramidate or a mixed phosphoramidate-phosphodiester oligomer. A polynucleotide may comprise modified nucleotides, such as methylated nucleotides and nucleotide analogs, uracyl, other sugars, and linking groups such as fluororibose and thioate, and nucleotide branches. The sequence of nucleotides may be interrupted by non-nucleotide components. A polynucleotide may be further modified after polymerization, such as by conjugation with a labeling component. Other types of modifications included in this definition are caps, substitution of one or more of the naturally occurring nucleotides with an analog, and introduction of means for attaching the polynucleotide to proteins, metal ions, labeling components, other polynucleotides, or a solid support. The term “polynucleotide” also encompasses peptidic nucleic acids, PNA and LNA. Polynucleotides may further comprise genomic DNA, cDNA, or DNA-RNA hybrids.

In the context of formation of a CRISPR complex, “target sequence” or “target nucleic acid” refers to a sequence to which a guide sequence is designed to have complementarity, where hybridization between a target sequence and a guide sequence promotes the formation of a CRISPR complex. A target sequence may comprise RNA polynucleotides. In some embodiments, a target sequence is located in the nucleus or cytoplasm of a cell. In one embodiment, the “target sequence” is a viral nucleic acid sequence. In one embodiment, the “target sequence” is an Enterovirus nucleic acid sequence.

The term “at least 80% sequence identity” includes at least 81% to 99% and all integer percentages there between.

The recitations “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I, U) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. Included are nucleotides and polypeptides having at least about 50%, 55%, 60%, 65%, 70%, 75%, 80%, 85%, 90%, 95%, 96%, 97%, 98%, 99% or 100% sequence identity to any of the reference sequences described herein, typically where the polypeptide variant maintains at least one biological activity of the reference polypeptide.

Terms used to describe sequence relationships between two or more polynucleotides or polypeptides include “reference sequence” “comparison window” “sequence identity.” “percentage of sequence identity,” and “substantial identity”. A “reference sequence” is at least 12 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, WI, USA) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25:3389. A detailed discussion of sequence analysis can be found in Unit 19.3 of Ausubel et al., Current Protocols in Molecular Biology, John Wiley & Sons Inc., 1994-1998, Chapter 15.

“Stringency conditions” refers to conditions under which a nucleic acid may hybridize to its target polynucleotide sequence. Preferably, under stringent conditions the nucleic acid hybridizes to its target polynucleotide sequence, but not other sequences. That is under stringent conditions, hybridisation is specific for the target sequence. Stringent conditions are sequence-dependent (e.g., longer sequences hybridize specifically at higher temperatures). Generally, stringent conditions are selected to be about 5° C. lower than the thermal melting point (Tm) for the specific sequence at a defined ionic strength and pH. The Tm is the temperature (under defined ionic strength, pH, and polynucleotide concentration) at which 50% of the probes complementary to the target sequence hybridize to the target sequence at equilibrium. Typically, stringent conditions will be those in which the salt concentration is at least about 0.01 to about 1.0 M sodium ion concentration (or other salts) at about pH 7.0 to about pH 8.3 and the temperature is at least about 30° C. for short probes (e.g., 10 to 50 nucleotides).

The first nucleic sequence (or spacer sequence) can be modified to hybridize to any desired sequence within a target nucleic acid. The first nucleic acid sequence can have a length from about 10 nucleotides to about 100 nucleotides. For example, it can have a length of from about 10 nucleotides (nt) to about 90 nt, from about 10 nt to about 80 nt, from about 10 nt to about 70 nt, from about 10 nt to about 60 nt, from about 10 nt to about 50 nt, from about 10 nt to about 40 nt, from about 10 nt to about 30 nt. For example, it can have a length of about 14 nt to about 30 nt, about 15 nt to about 30 nt, about 16 nt to about 30 nt, about 17 nt to about 30 nt, about 18 nt to about 30 nt, about 19 nt to about 30 nt, about 20 nt to about 30 nt, about 21 nt to about 30 nt, about 22 nt to about 30 nt or about 23 nt to about 30 nt. For example, it can have a length of from about 23 nt to about 30 nt, 23 nt to about 29 nt, from about 23 nt to about 28 nt, from about 23 nt to about 27 nt, from about 23 nt to about 26 nt, from about 23 nt to about 25 nt, or from about 23 nt to about 24 nt. The first nucleic acid sequence can be 14 nt, 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, 30 nt or more in length.

In one embodiment, the guide RNA is complementary to a viral nucleic acid or a variant thereof. The viral nucleic acid may, for example, be between 10 and 30 nucleotides in length. In one embodiment, the viral nucleic acid is a single stranded RNA. In one embodiment, the viral nucleic acid is a genomic RNA or a genomic RNA fragment. In another embodiment, the viral nucleic acid is a RNA transcript or RNA transcript fragment from Enterovirus.

In one embodiment, the guide RNA is complementary to an Enterovirus nucleic acid or a variant thereof. The Enterovirus nucleic acid may, for example, be between 10 and 30 nucleotides in length. In one embodiment, the Enterovirus nucleic acid is a single stranded RNA. In one embodiment, the Enterovirus nucleic acid is a genomic RNA or a genomic RNA fragment from Enterovirus. In another embodiment, the Enterovirus nucleic acid is a RNA transcript or RNA transcript fragment from Enterovirus. The Enterovirus nucleic acid may comprise or consist of a gene or corresponding mRNA from Enterovirus such as Enterovirus 2A protease (2A), Enterovirus viral protein 3 (VP3) and 3D polymerase (3Dpol).

In one embodiment, there is provided a guide RNA comprising a first nucleic acid sequence complementary to a viral nucleic acid and a second nucleic acid sequence capable of facilitating binding of a Cas13 nuclease to the viral nucleic acid.

In one embodiment, there is provided a guide RNA comprising a first nucleic acid sequence complementary to a viral nucleic acid and a second nucleic acid sequence capable of facilitating binding of a Cas13 nuclease to the viral nucleic acid. The first nucleic acid sequence may have at least 70% sequence identity to a nucleic acid sequence encoded by one of the nucleic acid sequences set forth in SEQ ID NO: 1-10. The first nucleic acid may have at least 70% sequence identity to one of the nucleic acid sequences set forth in SEQ ID NO: 11-20.

The first nucleic acid sequence may be contiguous with (or positioned adjacent to) the second nucleic acid sequence. Alternatively, the first nucleic acid sequence may be joined to the second nucleic acid sequence by a linker sequence.

In one embodiment, the guide RNA is chemically modified. Examples of guide RNA chemical modifications include, without limitation, incorporation of 2′-0-methyl (M), 2′-0-methyl 3 ‘phosphorothioate (MS), S-constrained ethyl (cEt), or 2’-0-methyl 3′thioPACE (MSP) at one or more terminal nucleotides. Such chemically modified guide RNAs can comprise increased stability and increased activity as compared to unmodified guide RNAs. Chemically modified guide RNAs further include, without limitation, RNAs with phosphorothioate linkages and locked nucleic acid (LNA) nucleotides comprising a methylene bridge between the 2′ and 4′ carbons of the ribose ring.

The first nucleic acid sequence may be positioned upstream or downstream of the second nucleic acid sequence.

In one embodiment, the gRNAs of the present invention comprise non-naturally occurring nucleic acids and/or non-naturally occurring nucleotides and/or nucleotide analogs, and/or chemically modifications. Non-naturally occurring nucleic acids can include, for example, mixtures of naturally and non-naturally occurring nucleotides. Non-naturally occurring nucleotides and/or nucleotide analogs may be modified at the ribose, phosphate, and/or base moiety. In an embodiment of the invention, a guide nucleic acid comprises ribonucleotides and non-ribonucleotides. In one embodiment, a guide RNA comprises one or more ribonucleotides and one or more deoxyribonucleotides. In one embodiment, the guide RNA comprises one or more non-naturally occurring nucleotide or nucleotide analog such as a nucleotide with phosphorothioate linkage, boranophosphate linkage, a locked nucleic acid (LNA) nucleotides comprising a methylene bridge between the 2′ and 4′ carbons of the ribose ring, or bridged nucleic acids (BNA). Other examples of modified nucleotides include 2′-0-methyl analogs, 2′-deoxy analogs, 2-thiouridine analogs, N6-methyladenosine analogs, or 2′-fluoro analogs. Further examples of modified bases include, but are not limited to, 2-aminopurine, 5-bromo-uridine, pseudouridine (Ψ), NI-methylpseudouridine (me1Ψ), S-methoxyuridine (SmoU), inosine, 7-methylguanosine.

### Treatment

The present specification is directed to a method of preventing or treating a virus infection in a subject.

The term “treating” as used herein may refer to (1) preventing or delaying the appearance of one or more symptoms of the disorder: (2) inhibiting the development of the disorder or one or more symptoms of the disorder: (3) relieving the disorder, i.e., causing regression of the disorder or at least one or more symptoms of the disorder; and/or (4) causing a decrease in the severity of one or more symptoms of the disorder.

The term “administering” refers to contacting, applying, injecting, transfusing or providing a composition of the present invention to a subject.

The term “subject” as used throughout the specification is to be understood to mean a human or may be a domestic or companion animal. While it is particularly contemplated that the methods of the invention are for treatment of humans, they are also applicable to veterinary treatments, including treatment of companion animals such as dogs and cats, and domestic animals such as horses, cattle and sheep, or zoo animals such as primates, felids, canids, bovids, and ungulates. The “subject” may include a person, a patient or individual, and may be of any age or gender. The “subject” may be a pediatric subject.

The methods as defined herein may comprise administering an effective amount of a administering a recombinant adeno-associated virus (AAV) to the subject. In one embodiment, the method comprises administering a dose of about 5×10¹¹ to about 1×10¹³ vector genomes (vgs)/kg of a recombinant adeno-associated virus (AAV) to the subject.

The term “effective amount” as defined herein is meant the administration of an amount of agent to an individual in need thereof, either in a single dose or as part of a series that is effective for that elicitation, treatment or prevention. The effective amount will vary depending upon the health and physical condition of the individual to be treated, the taxonomic group of individual to be treated, the formulation of the composition, the assessment of the medical situation, and other relevant factors. It is expected that the amount will fall in a relatively broad range that can be determined through routine trials.

### Nucleic Acids/Constructs

Provided herein is a nucleic acid comprising or encoding a guide RNA as defined herein.

Provided herein is a construct comprising a nucleic acid encoding a guide RNA. The construct may further comprise a nucleic acid encoding a Cas nuclease.

Disclosed herein is a vector comprising a nucleic acid sequence encoding one or more guide RNAs as defined herein. The vector may further comprise a nucleic acid encoding a Cas nuclease. The vector may be designed to target the viral genome at multiple locations, thereby increasing the chance of incapacitating the virus within a cell.

The term “construct” refers to a recombinant genetic molecule including one or more isolated nucleic acid sequences from different sources. Thus, constructs are chimeric molecules in which two or more nucleic acid sequences of different origin are assembled into a single nucleic acid molecule and include any construct that contains (1) nucleic acid sequences, including regulatory and coding sequences that are not found together in nature (i.e., at least one of the nucleotide sequences is heterologous with respect to at least one of its other nucleotide sequences), or (2) sequences encoding parts of functional RNA molecules or proteins not naturally adjoined, or (3) parts of promoters that are not naturally adjoined. Representative constructs include any recombinant nucleic acid molecule such as a plasmid, cosmid, virus, autonomously replicating polynucleotide molecule, phage, or linear or circular single stranded or double stranded DNA or RNA nucleic acid molecule, derived from any source, capable of genomic integration or autonomous replication, comprising a nucleic acid molecule where one or more nucleic acid molecules have been operably linked. Constructs of the present invention will generally include the necessary elements to direct expression of a nucleic acid sequence of interest that is also contained in the construct, such as, for example, a guide RNA sequence. Such elements may include control elements such as a promoter that is operably linked to (so as to direct transcription of) the nucleic acid sequence of interest, and often includes a polyadenylation sequence as well. Within certain embodiments of the invention, the construct may be contained within a vector. In addition to the components of the construct, the vector may include, for example, one or more selectable markers, one or more origins of replication, such as prokaryotic and eukaryotic origins, at least one multiple cloning site, and/or elements to facilitate stable integration of the construct into the genome of a host cell. Two or more constructs can be contained within a single nucleic acid molecule, such as a single vector, or can be containing within two or more separate nucleic acid molecules, such as two or more separate vectors. An “expression construct” generally includes at least a control sequence operably linked to a nucleotide sequence of interest. In this manner, for example, promoters in operable connection with the nucleotide sequences to be expressed are provided in expression constructs for expression in an organism or part thereof including a host cell. For the practice of the present invention, conventional compositions and methods for preparing and using constructs and host cells are well known to one skilled in the art, see for example, Molecular Cloning: A Laboratory Manual, 3rd edition Volumes 1, 2, and 3. J. F. Sambrook, D. W. Russell, and N. Irwin, Cold Spring Harbor Laboratory Press, 2000.

A used herein, a “vector” is a tool that allows or facilitates the transfer of an entity from one environment to another. In general, the term “vector” refers to a nucleic acid molecule capable of transporting another nucleic acid to which it has been linked. Vectors include, but are not limited to, nucleic acid molecules that are single-stranded, double-stranded, or partially double-stranded: nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DMA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses (AAVs)). Viral vectors also include polynucleotides carried by a virus or viral-like particles for transduction into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

In some embodiments, the vector is circular. In other embodiments, the vector is linear. In some embodiments, the vector is enclosed in a lipid nanoparticle, liposome, non-lipid nanoparticle, or viral capsid. Non-limiting exemplary vectors include plasmids, phagemids, cosmids, artificial chromosomes, minichromosomes, transposons, viral vectors, and expression vectors.

As used herein, the terms “encode,” “encoding” and the like refer to the capacity of a nucleic acid to provide for another nucleic acid or a polypeptide. For example, a nucleic acid sequence is said to “encode” a polypeptide if it can be transcribed and/or translated to produce the polypeptide or if it can be processed into a form that can be transcribed and/or translated to produce the polypeptide. Such a nucleic acid sequence may include a coding sequence or both a coding sequence and a non-coding sequence. Thus, the terms “encode,” “encoding” and the like include an RNA product resulting from transcription of a DNA molecule, a protein resulting from translation of an RNA molecule, a protein resulting from transcription of a DNA molecule to form an RNA product and the subsequent translation of the RNA product, or a protein resulting from transcription of a DNA molecule to provide an RNA product, processing of the RNA product to provide a processed RNA product (e.g., mRNA) and the subsequent translation of the processed RNA product.

“Polypeptide,” “peptide” and “protein” are used interchangeably herein to refer to molecules comprising or consisting of a polymer of amino acid residues and to variants and synthetic analogues of the same. Thus, these terms apply to amino acid polymers in which one or more amino acid residues are synthetic non-naturally occurring amino acids, such as a chemical analogue of a corresponding naturally occurring amino acid, as well as to naturally-occurring amino acid polymers.

As used herein the term “recombinant” as applied to “nucleic acid molecules,” “polynucleotides” and the like is understood to mean artificial nucleic acid structures (i.e., non-replicating cDNA or RNA: or replicons, self-replicating cDNA or RNA) which can be transcribed and/or translated in host cells or cell-free systems described herein. Recombinant nucleic acid molecules or polynucleotides may be inserted into a vector. Non-viral vectors such as plasmid expression vectors or viral vectors may be used. The kind of vectors and the technique of insertion of the nucleic acid construct would be known to persons skilled in the art. A nucleic acid molecule or polynucleotide according to this disclosure does not occur in nature in the arrangement described by the present invention. In other words, a heterologous nucleotide sequence is not naturally combined with elements of a parent virus genome (e.g., promoter, ORF, polyadenylation signal, DNA-recognition moiety, endonuclease).

The term “operably connected” or “operably linked” as used herein refers to a juxtaposition wherein the components so described are in a relationship permitting them to function in their intended manner. For example, a regulatory element or regulatory sequence “operably linked” to a coding sequence refers to positioning and/or orientation of the regulatory sequence relative to the coding sequence to permit expression of the coding sequence under conditions compatible with the regulatory sequence.

The control sequences need not be contiguous with the nucleotide sequence of interest, as long as they function to direct the expression thereof. Thus, for example, intervening untranslated, yet transcribed, sequences can be present between a promoter and a coding sequence, and the promoter sequence can still be considered “operably linked” to the coding sequence.

By “regulatory element”, “regulatory sequence”, “control element” or “control sequence” is meant nucleic acid sequences (e.g., DNA) necessary for expression of an operably linked coding sequence in a particular host cell. The regulatory sequences that are suitable for eukaryotic cells include promoters, polyadenylation signals, transcriptional enhancers, translational enhancers, leader or trailing sequences that modulate mRNA stability, as well as targeting sequences that target a product encoded by a transcribed polynucleotide to an intracellular compartment within a cell or to the extracellular environment.

“Promoter” refers to a nucleotide sequence, usually upstream (5′) to its coding sequence, which controls the expression of the coding sequence by providing the recognition for RNA polymerase and other factors required for proper transcription. “Promoter regulatory sequences” consist of proximal and more distal upstream elements. Promoter regulatory sequences influence the transcription, RNA processing or stability, or translation of the associated coding sequence. Regulatory sequences include enhancers, promoters, untranslated leader sequences, introns, and polyadenylation signal sequences. They include natural and synthetic sequences as well as sequences that may be a combination of synthetic and natural sequences. An “enhancer” is a DNA sequence that can stimulate promoter activity and may be an innate element of the promoter or a heterologous element inserted to enhance the level or tissue specificity of a promoter. It is capable of operating in both orientations (normal or flipped), and is capable of functioning even when moved either upstream or downstream from the promoter. The meaning of the term “promoter” includes “promoter regulatory sequences.”

In an embodiment, the regulatory element is a promoter.

The terms “open reading frame” and “ORF” refer to the amino acid sequence encoded between translation initiation and termination codons of a coding sequence. The terms “initiation codon” and “termination codon” refer to a unit of three adjacent nucleotides (‘codon’) in a coding sequence that specifies initiation and chain termination, respectively, of protein synthesis (mRNA translation).

### Delivery

In some embodiments, the present invention involve introducing or delivering a guide RNA or vector as defined herein, or any of the therapeutic agents described herein, into a cell of interest. It should be appreciated that agents can be introduced into cells in an in vitro model or an in vivo model.

In some embodiments, the guide RNA or vector as defined herein can be transfected into cells by various methods, including viral vectors and non-viral vectors. Viral vectors may include retroviruses, lentiviruses, adenoviruses, and adeno-associated viruses (AAV). It should be appreciated that any viral vector may be incorporated into the present invention to effectuate delivery of the guide RNA or vector into a cell. Some viral vectors may be more effective than others, depending on the guide RNA or vector designed for digestion or incapacitation. In one embodiment, the vectors contain essential components such as origin of replication, which is necessary for the replication and maintenance of the vector in the host cell.

A retrovirus is a single-stranded RNA virus that stores its nucleic acid in the form of an mRNA genome (including the 5′ cap and 3′ Poly A tail) and targets a host cell as an obligate parasite. In some methods in the art, retroviruses have been used to introduce nucleic acids into a cell. Once inside the host cell cytoplasm the virus uses its own reverse transcriptase enzyme to produce DNA from its RNA genome, the reverse of the usual pattern, thus retro (backwards). This new DNA is then incorporated into the host cell genome by an integrase enzyme, at which point the retroviral DNA is referred to as a provirus. For example, the recombinant retroviruses such as the Moloney murine leukemia virus have the ability to integrate into the host genome in a stable fashion. They contain a reverse transcriptase that allows integration into the host genome. Retroviral vectors can either be replication-competent or replication-defective. In some embodiments, retroviruses are incorporated to effectuate transfection into a cell, however the Cas/gRNA complexes are designed to target the viral genome.

In some embodiments of the invention, lentiviruses, which are a subclass of retroviruses, are used as viral vectors. Lentiviruses can be adapted as delivery vehicles (vectors) given their ability to integrate into the genome of non-dividing cells, which is the unique feature of lentiviruses as other retroviruses can infect only dividing cells. The viral genome in the form of RNA is reverse-transcribed when the virus enters the cell to produce DNA, which is then inserted into the genome at a random position by the viral integrase enzyme. The vector, now called a provirus, remains in the genome and is passed on to the progeny of the cell when it divides.

As opposed to lentiviruses, adenoviral DNA does not integrate into the genome and is not replicated during cell division. Adenovirus and the related AAV can be used as delivery vectors since they do not integrate into the host's genome. In some aspects of the invention, only the viral genome to be targeted is effected by the Cas/gRNA complex, and not the host's cells. Adeno-associated virus (AAV) is a small virus that infects humans and some other primate species. AAV can infect both dividing and non-dividing cells and may incorporate its genome into that of the host cell. For example, because of its potential use as a gene therapy vector, researchers have created an altered AAV called self-complementary adeno-associated virus (scAAV). Whereas AAV packages a single strand of DNA and requires the process of second-strand synthesis, scAAV packages both strands which anneal together to form double stranded DNA. By skipping second strand synthesis scAAV allows for rapid expression in the cell. Otherwise, scAAV carries many characteristics of its AAV counterpart. Methods of the invention may incorporate herpesvirus, poxvirus, alphavirus, or vaccinia virus as a means of delivery vectors.

Non-limiting exemplary viral vectors include adeno-associated virus (AAV) vector, lentivirus vectors, adenovirus vectors, helper dependent adenoviral vectors (HDAd), herpes simplex virus (HSV-1) vectors, bacteriophage T4, baculovirus vectors, and retrovirus vectors. In some embodiments, the viral vector is an AAV vector. The AAV vector may be AAV type 1 (AAV-1), AAV type 2 (AAV-2), AAV type 3 (AAV-3), AAV type 4 (AAV-4), AAV type 5 (AAV-5), AAV type 6 (AAV-6), AAV type 7 (AAV-7), AAV type 8 (AAV-8), AAV9, AAV10, AAV11, AAV12, AAV13, rh10, AAVDJ, AAV-PHP.S, AAV-PHP.B. AAVPHP.eB, and Anc80. The AAV vector may be AAV2, AAVDJ or AAV1.

In some embodiments, the vector comprises a nucleotide sequence encoding a Cas nuclease. In some embodiments, the nuclease encoded by the vector may be a Cas protein. In some embodiments, the vector system comprises one copy of the nucleotide sequence encoding the nuclease. In other embodiments, the vector system comprises more than one copy of the nucleotide sequence encoding the nuclease. In some embodiments, the nucleotide sequence encoding the nuclease is operably linked to at least one transcriptional or translational control sequence. In some embodiments, the nucleotide sequence encoding the nuclease is operably linked to at least one promoter.

In some embodiments, the promoter is constitutive or inducible. In some embodiments, the promoter may be a constitutive promoter. Non-limiting exemplary constitutive promoters include cytomegalovirus immediate early promoter (CMV), simian virus (SV40) promoter, adenovirus major late (MLP) promoter, Rous sarcoma virus (RSV) promoter, mouse mammary tumor virus (MMTV) promoter, phosphoglycerate kinase (PGK) promoter, elongation factor-alpha (EF1a) promoter, ubiquitin promoters, actin promoters, tubulin promoters, immunoglobulin promoters, a functional fragment thereof, or a combination of any of the foregoing. In some embodiments, the promoter is a CMV promoter. In some embodiments, the promoter is a truncated CMV promoter. In other embodiments, the promoter may be an EF1a promoter. In some embodiments, the promoter is an inducible promoter. Non-limiting exemplary inducible promoters include those inducible by heat shock, light, chemicals, peptides, metals, steroids, antibiotics, or alcohol. In some embodiments, the inducible promoter is one that has a low basal (non-induced) expression level, such as, e.g., the Tet-On® promoter (Clontech).

The vector may comprise a nucleotide sequence encoding the guide RNA described herein. In some embodiments, the vector comprises one copy of the guide RNA. In other embodiments, the vector comprises more than one copy of the guide RNA. In embodiments with more than one guide RNA, the guide RNAs may be non-identical such that they target different target sequences, or may be identical in that they target the same target sequence. In some embodiments where the vectors comprise more than one guide RNA, each guide RNA may have other different properties, such as activity or stability within a complex with a Cas nuclease. In some embodiments, the nucleotide sequence encoding the guide RNA may be operably linked to at least one transcriptional or translational control sequence, such as a promoter, a 3′ UTR, or a 5′ UTR. In one embodiment, the promoter may be a tRNA promoter, e.g., tRNA^{Lys3}, or a tRNA chimera. In some embodiments, the promoter may be recognized by RNA polymerase III (Pol III). Non-limiting examples of Pol III promoters include U6 and H1 promoters. In some embodiments, the nucleotide sequence encoding the guide RNA may be operably linked to a mouse or human U6 promoter. In other embodiments, the nucleotide sequence encoding the guide RNA may be operably linked to a mouse or human H1 promoter. In embodiments with more than one guide RNA, the promoters used to drive expression may be the same or different.

In some embodiments, the nucleotide sequence encoding the guide RNA may be located on the same vector comprising the nucleotide sequence encoding a Cas nuclease. In some embodiments, expression of the guide RNA and of the Cas nuclease may be driven by their own corresponding promoters. In some embodiments, expression of the guide RNA may be driven by the same promoter that drives expression of the Cas nuclease such as a Cas protein. In some embodiments, the guide RNA and the Cas nuclease such as a Cas protein transcript may be contained within a single transcript. For example, the guide RNA may be within an untranslated region (UTR) of the Cas nuclease such as a Cas protein transcript. In some embodiments, the guide RNA may be within the 5′ UTR of the transcript. In other embodiments, the guide RNA may be within the 3′ UTR of the transcript. In some embodiments, the intracellular half-life of the transcript may be reduced by containing the guide RNA within its 3′ UTR and thereby shortening the length of its 3′ UTR. In additional embodiments, the guide RNA may be within an intron of the transcript. In some embodiments, suitable splice sites may be added at the intron within which the guide RNA is located such that the guide RNA is properly spliced out of the transcript. In some embodiments, expression of the Cas nuclease such as a Cas protein and the guide RNA from the same vector in close temporal proximity may facilitate more efficient formation of the CRISPR RNP complex.

In some embodiments, the compositions comprise a vector system. In some embodiments, the vector system may comprise one single vector. In other embodiments, the vector system may comprise two vectors. In additional embodiments, the vector system may comprise three vectors. When different guide RNAs are used for multiplexing, or when multiple copies of the guide RNA are used, the vector system may comprise more than three vectors.

In certain embodiments of the invention, non-viral vectors may be used to effectuate transfection. Methods of non-viral delivery of nucleic acids include lipofection, nucleofection, microinjection, biolistics, virosomes, liposomes, micelles, immunoliposomes, polycation or lipid: nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam and Lipofectin). Cationic and neutral lipids that are suitable for efficient receptor-recognition lipofection of polynucleotides include those in U.S. Pat. No. 7,166,298 or U.S. Pat. No. 6,890,554.

The CRISPR enzyme and/or any of the present RNAs, for instance a guide RNA, can be delivered using any suitable vector, e.g., plasmid or viral vectors, such as adeno associated virus (AAV), lentivirus, adenovirus or other viral vector types, or combinations thereof. Effector proteins and one or more guide RNAs can be packaged into one or more vectors, e.g., plasmid or viral vectors. In some embodiments, the vector, e.g., plasmid or viral vector is delivered by, for example, an intramuscular injection, while other times the delivery is via intravenous, transdermal, intranasal, oral, mucosal, or other delivery methods. In some embodiments, the vector is delivered intravenously, intraperitoneally or orally to the subject. Such delivery may be either via a single dose, or multiple doses. One skilled in the art understands that the actual dosage to be delivered herein may vary greatly depending upon a variety of factors, such as the vector choice, the target cell, organism, or tissue, the general condition of the subject to be treated, the degree of transformation/modification sought, the administration route, the administration mode, the type of transformation/modification sought, etc.

Such a dosage may further contain, for example, a carrier (water, saline, ethanol, glycerol, lactose, sucrose, calcium phosphate, gelatin, dextran, agar, pectin, peanut oil, sesame oil, etc.), a diluent, a pharmaceutically-acceptable carrier (e.g., phosphate-buffered saline), a pharmaceutically-acceptable excipient, and/or other compounds known in the art. The dosage may further contain one or more pharmaceutically acceptable salts such as, for example, a mineral acid salt such as a hydrochloride, a hydrobromide, a phosphate, a sulfate, etc.; and the salts of organic acids such as acetates, propionates, malonates, benzoates, etc. Additionally, auxiliary substances, such as wetting or emulsifying agents, pH buffering substances, gels or gelling materials, flavorings, colorants, microspheres, polymers, suspension agents, etc. may also be present herein. In addition, one or more other conventional pharmaceutical ingredients, such as preservatives, humectants, suspending agents, surfactants, antioxidants, anti caking agents, fillers, chelating agents, coating agents, chemical stabilizers, etc. may also be present, especially if the dosage form is a reconstitutable form. Suitable exemplary ingredients include microcrystalline cellulose, carboxymethylcellulose sodium, polysorbate 80, phenylethyl alcohol, chlorobutanol, potassium sorbate, sorbic acid, sulfur dioxide, propyl gallate, the parabens, ethyl vanillin, glycerin, phenol, parachlorophenol, gelatin, albumin and a combination thereof. A thorough discussion of pharmaceutically acceptable excipients is available in REMINGTON'S PHARMACEUTICAL SCIENCES (Mack Pub. Co., N.J. 1991) which is incorporated by reference herein.

### Dosage

In an embodiment herein, the delivery is via an AAV. The dosage may be adjusted to balance the therapeutic benefit against any side effects. In one embodiment, the AAV may be administered at a dose of about 5×10¹¹ to about 5×10¹³ vgs/kg.

Without being bound by theory, the inventors have found that the AAV dosages used in the present invention are both safe and efficacious in preventing or treating an RNA viral infection in a subject.

In one embodiment, the AAV is administered at a dose of about 5×10¹² vgs/kg to about 5×10¹³ vgs/kg. For example, the AAV may be administered at a dose of about 5×10¹² vgs/kg, about 6×10¹² vgs/kg, about 7×10¹² vgs/kg, about 8×10¹² vgs/kg, about 9×10¹² vgs/kg, about 1×10¹³ vgs/kg, about 2×10¹³ vgs/kg, about 3×10¹³ vgs/kg, about 4×10¹³ vgs/kg or about 5×10¹³ vgs/kg. In one embodiment, the AAV is administered at a dose of about 5×10¹² vgs/kg to about 6×10¹² vgs/kg. In one embodiment, the AAV is administered at a dose of about 6×10¹² vgs/kg to about 7×10¹² vgs/kg. In one embodiment, the AAV is administered at a dose of about 7×10¹² vgs/kg to about 8×10¹² vgs/kg. In one embodiment, the AAV is administered at a dose of about 8×10¹² vgs/kg to about 9×10¹² vgs/kg. In one embodiment, the AAV is administered at a dose of about 9×10¹² vgs/kg to about 1×10¹³ vgs/kg. In one embodiment, the AAV is administered at a dose of about 1×10¹³ vgs/kg to about 2×10¹³ vgs/kg. In one embodiment, the AAV is administered at a dose of about 2×10¹³ vgs/kg to about 3×10¹³ vgs/kg. In one embodiment, the AAV is administered at a dose of about 3×10¹³ vgs/kg to about 4×10¹³ vgs/kg. In one embodiment, the AAV is administered at a dose of about 4×10¹³ vgs/kg to about 5×10¹³ vgs/kg.

In one embodiment, the AAV is administered at a dose of about 5×10¹¹ vgs/kg to about 5×10¹² vgs/kg. For example, the AAV may be administered at a dose of about 5×10¹¹ vgs/kg, about 6×10¹¹ vgs/kg, about 7×10¹¹ vgs/kg, about 8×10¹¹ vgs/kg, about 9×10¹¹ vgs/kg, about 1×10¹² vgs/kg, about 2×10¹² vgs/kg, about 3×10¹² vgs/kg, about 4×10¹² vgs/kg or about 5×10¹² vgs/kg. In one embodiment, the AAV is administered at a dose of about 5×10¹¹ vgs/kg to about 6×10¹¹ vgs/kg. In one embodiment, the AAV is administered at a dose of about 6×10¹¹ vgs/kg to about 7×10¹¹ vgs/kg. In one embodiment, the AAV is administered at a dose of about 7×10¹¹ vgs/kg to about 8×10¹¹ vgs/kg. In one embodiment, the AAV is administered at a dose of about 8×10¹¹ vgs/kg to about 9×10¹¹ vgs/kg. In one embodiment, the AAV is administered at a dose of about 9×10¹¹ vgs/kg to about 1×10¹² vgs/kg. In one embodiment, the AAV is administered at a dose of about 1×10¹² vgs/kg to about 2×10¹² vgs/kg. In one embodiment, the AAV is administered at a dose of about 2×10¹² vgs/kg to about 3×10¹² vgs/kg. In one embodiment, the AAV is administered at a dose of about 3×10¹² vgs/kg to about 4×10¹² vgs/kg. In one embodiment, the AAV is administered at a dose of about 4×10¹² vgs/kg to about 5×10¹² vgs/kg.

In an alternative embodiment, the AAV is administered at a dose of about 5×10⁹ vgs/kg to about 5×10¹⁰ vgs/kg. For example, the AAV may be administered at a dose of about 5×10⁹ vgs/kg, about 6×10⁹ vgs/kg, about 7×10⁹ vgs/kg, about 8×10⁹ vgs/kg, about 9×10⁹ vgs/kg, about 1×10¹⁰ vgs/kg, about 2×10¹⁰ vgs/kg, about 3×10¹⁰ vgs/kg, about 4×10¹⁰ vgs/kg or about 5×10¹⁰ vgs/kg.

In an alternative embodiment, the AAV is administered at a dose of about 5×10¹⁰ vgs/kg to about 5×10¹¹ vgs/kg. For example, the AAV may be administered at a dose of about 5×10¹⁰ vgs/kg, about 6×10¹⁰ vgs/kg, about 7×10¹⁰ vgs/kg, about 8×10¹⁰ vgs/kg, about 9×10¹⁰ vgs/kg, about 1×10¹¹ vgs/kg, about 2×10¹¹ vgs/kg, about 3×10¹¹ vgs/kg, about 4×10¹¹ vgs/kg or about 5×10¹¹ vgs/kg.

Such concentrations may be delivered in from about 0.001 ml to about 100 ml, about 0.05 to about 50 ml, or about 10 to about 25 ml of a carrier solution.

The AAV may be administered within 1 h, 2 h, 3 h, 4 h, 6 h, 7 h, 8 h, 9 h, 10 h, 11 h, 12 h, 13 h, 14 h, 15 h, 16 h, 17 h, 18 h, 19 h, 20 h, 21 h, 22 h, 23 h, 24 h, 2 days, 3 days, 4 days, 5 days, 6 days or 7 days following infection in a subject.

In one embodiment, the method as defined herein comprises inhibiting the RNA viral nucleic acid in a skeletal or central nervous system (CNS) cell or tissue of the subject. Without being bound by theory, it was found that a recombinant adeno-associated virus (AAV) as defined herein is capable of direct elimination of EV-A71 from skeletal muscle or central nervous system (CNS), which are important reservoirs and key tissues for EV-A71 tropism and pathology. The method may comprise administering about 5×10¹¹ to about 5×10¹³ vgs/kg of a recombinant adeno-associated virus (AAV) to the subject.

In one embodiment, there is provided a method of inhibiting the RNA viral nucleic acid in a skeletal or central nervous system (CNS) cell or tissue of a subject, the method comprising administering a recombinant adeno-associated virus (AAV) to the subject, wherein the AAV comprises at least one heterologous nucleic acid sequence encoding a Cas13 nuclease and one or more guide RNAs. The method may comprise administering about 5×10¹¹ to about 5×10¹³ vgs/kg of a recombinant adeno-associated virus (AAV) to the subject. The method may be capable of preventing or treating an RNA viral infection in the subject.

Disclosed herein is a method of inhibiting a viral nucleic acid in a subject, the method comprising administering about 5×10¹¹ to about 5×10¹³ vgs/kg of a recombinant adeno-associated virus (AAV) to the subject, wherein the AAV comprises at least one heterologous nucleic acid sequence encoding a Cas13 nuclease and one or more guide RNAs. In one embodiment, the method comprises inhibiting the RNA viral nucleic acid in a skeletal or central nervous system (CNS) cell or tissue of the subject. Without being bound by theory, it was found that about 5×10¹¹ to about 5×10¹³ vgs/kg of a recombinant adeno-associated virus (AAV) as defined herein is capable of direct elimination of EV-A71 from skeletal muscle or central nervous system (CNS), which are important reservoirs and key tissues for EV-A71 tropism and pathology.

In one embodiment, there is provided a recombinant adeno-associated virus (AAV) for use in inhibiting a viral nucleic acid in a subject, wherein about 5×10¹¹ to about 5×10¹³ vgs/kg of the recombinant AAV is to be administered to the subject, wherein the AAV comprises at least one heterologous nucleic acid sequence encoding a Cas13 nuclease and one or more guide RNAs.

In one embodiment, there is provided a use of a recombinant adeno-associated virus (AAV) in the manufacture of a medicament for inhibiting a viral nucleic acid in a subject, wherein about 5×10¹¹ to about 5×10¹³ vgs/kg of the recombinant AAV is to be administered to the subject, wherein the AAV comprises at least one heterologous nucleic acid sequence encoding a Cas13 nuclease and one or more guide RNAs.

Disclosed herein is a recombinant adeno-associated virus (AAV) for use in treating a virus infection in a subject, wherein about 5×10¹¹ to about 5×10¹³ vgs/kg of the recombinant AAV is to be administered to the subject, wherein the AAV comprises at least one heterologous nucleic acid sequence encoding a Cas13 nuclease and one or more guide RNAs.

Disclosed herein is the use of a recombinant adeno-associated virus (AAV) in the manufacture of a medicament for treating a virus infection in a subject, wherein about 5×10¹¹ to about 5×10¹³ vgs/kg of the recombinant AAV is to be administered to the subject, wherein the AAV comprises at least one heterologous nucleic acid sequence encoding a Cas13 nuclease and one or more guide RNAs.

Disclosed herein is a pharmaceutical composition comprising a recombinant adeno-associated virus (AAV) as defined herein. The pharmaceutical composition may comprise a pharmaceutically acceptable carrier.

In one embodiment, there is provided a pharmaceutical composition comprising a guide RNA, vector or construct as defined herein. The pharmaceutical composition may comprise a pharmaceutically acceptable carrier.

Compositions include, but are not limited to pharmaceutical compositions. A “pharmaceutical composition” refers to a composition formulated in pharmaceutically-acceptable or physiologically-acceptable solutions for administration to a cell or an animal, either alone, or in combination with one or more other modalities of therapy. It will also be understood that, if desired, the compositions may be administered in combination with other agents as well, such as, e.g., cytokines, growth factors, hormones, small molecules, chemotherapeutics, pro-drugs, drugs, antibodies, or other various pharmaceutically-active agents. There is virtually no limit to other components that may also be included in the compositions, provided that the additional agents do not adversely affect the composition.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

By “pharmaceutically acceptable carrier” is meant a solid or liquid filler, diluent or encapsulating substance that can be safely used in topical or systemic administration to an animal, preferably a mammal, including humans. Representative pharmaceutically acceptable carriers include any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient(s), its use in the pharmaceutical compositions is contemplated.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

By “about” is meant a quantity, level, value, number, frequency, percentage, dimension, size, amount, weight, position or length that varies by as much 10, 9, 8, 7, 6, 5, 4, 3, 2 or 1% to a reference quantity, level, value, number, frequency, percentage, dimension, size, amount, weight, position or length.

As used herein, “and/or” refers to and encompasses any and all possible combinations of one or more of the associated listed items, as well as the lack of combinations when interpreted in the alternative (or).

As used in this application, the singular form “a,” “an,” and “the” include plural references unless the context clearly dictates otherwise. For example, the term “an agent” includes a plurality of agents, including mixtures thereof.

Throughout this specification and the statements which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Those skilled in the art will appreciate that the invention described herein is susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

Unless otherwise defined, all technical and scientific terms used herein have the same meanings as commonly understood by one of ordinary skill in the art to which this invention belongs.

Certain embodiments of the invention will now be described with reference to the following examples which are intended for the purpose of illustration only and are not intended to limit the scope of the generality hereinbefore described.

### EXAMPLES

### Materials and Methods

### Design and Construction of AAV-Vector Based CRISPR-Cas Cargo Plasmid

The CasRx sequence was a gift from Patrick Hsu (pXR001: EF1a-CasRX-2A-EGFP, Addgene no. 109049). Using a plasmid backbone with AAV2 ITRs, EcoRI digestion was performed to create a cut between the two ITR sequences for insertion of the custom-designed expression cassette. First, the mammalian CMV promoter and enhancer sequence fragment were derived from PCR amplification using pAAV-SMVP-Cas9C. Then, CasRx with a HA tag and a rabbit poly A tail (CasRx-HA-polyA) downstream of the mammalian CMV promoter and enhancer was assembled into the AAV vector plasmid containing AAV2 ITR sequences by using Gibson reaction. Immediately downstream of the expression cassette, the gRNA backbone driven by human U6 promoter was assembled using Gibson reaction and the sequence was a gift from Patrick Hsu (pXR003: CasRx gRNA cloning backbone, Addgene no. 109053). The expression cassette is then assembled to the EcoRI-digested plasmid with AAV2 ITRs using Gibson assembly. Individual guide sequences derived from the bioinformatics analysis were cloned in using the BbsI digestion of the vector plasmid via Gibson assembly of the target guide sequences.

### Bioinformatics Guide Selection Pipeline (Cas13gRNAtor)

795 published EV-A71 complete genome assemblies from NCBI virus (https://www.ncbi.nlm.nih.gov/labs/virus/) were aligned using MAFFT and Cas13gRNAtor was run on these sequences. A bash script was created to align the input sequences using MAFFT command line. The length was kept constant to the reference genome. Conservation score at each nucleotide position was calculated as the percentage of sequences matching the consensus at that nucleotide position. A score of 1.0 indicates that the nucleotide position is fully conserved across all sequences. Shannon Entropy (SE) score calculated the propensity towards order and disorder for each nucleotide position³⁴. Entropy was directly proportional to the rate of disorder, ie. a higher SE score indicated higher disorder of that particular nucleotide position. Shannon Entropy is defined as follow: SE(i)=−Σᵢ₌₁ⁿ Pᵢ log₂ Pᵢ, where Pᵢ was the probability of a given nucleotide (A, C, G, T, N: where N is an unspecified or deleted nucleotide) and n was the number of sequences used in the alignment. The summation run over the 5 nucleotides in all the aligned sequences on every nucleotide position. The entropy range lied between 0 and the log 2(5)=2.32. A consensus sequence was generated based on the most conserved nucleotide at each position for downstream processing. In the next module within Cas13gRNAtor, Cas13gRNAtor utilized a model and package developed in Wessel et al., 2020 (https://gitlab.com/sanjanalab/cas13) to generate additional scores for all possible gRNAs for the consensus sequence. The random forest model here itself utilized a few independent software that includes RNAfold, a program to predict RNA secondary structure and minimum free energy (MFE), RNAplfold that measures RNA accessibility (Target RNA unpaired probability), and RNAhybrid that calculates RNA-RNA hybridization between the guide RNA and its target site and RNA-RNA hybridization MFEs for each gRNA nucleotide. Through this random forest model, based on the highest percentage increase in mean squared error (% incMSE), it was inferred that a few key features help predict Cas13d on-target efficiency, including the crRNA folding energy, the local target ‘C’-context and the upstream target ‘U’-context as the most important features. Cas13gRNAtor scores gRNAs according to these features. Cas13gRNAtor then filters for the best candidate gRNAs from the 4th score quartile. Candidate gRNAs having T-homopolymers (>3 Ts) sequences are also removed. For each predicted gRNAs, Cas13gRNAtor takes the entropy and conservation scores of their nucleotide positions in the consensus sequence (including in a more mutational intolerant ‘seed region’ aka ‘intolerant region’) and derives the means and standard deviations. The gRNA is considered conserved if the gRNA and seed region have a mean Conservation Score of >0.9. Cas13gRNAtor scores gRNAs according to these features.

### Cell Lines and Viruses

Human RD cells and mouse C2C12 cells were purchased from the American Type Culture Collection (ATCC). Human B4G12 cells were purchased from Creative Bioarray. The cells were grown in media recommended by ATCC and Creative Bioarray respectively. AAVs were generated in-house as previously reported. Briefly. AAV were packaged via a triple transfection of 293AAV cell line (Cell Biolabs AAV-100) that were plated in a HYPERFlask ‘M’ (Corning) in growth media consisting of DMEM+glutaMax+pyruvate+10% FBS (Thermo Fisher), supplemented with 1×MEM non-essential amino acids (Gibco). Confluency at transfection was between 70-90%. Media was replaced with fresh pre-warmed growth media before transfection. For each HYPERFlask ‘M’, 200 μg of pHelper (Cell Biolabs), 100 μg of pRepCap [encoding capsid proteins for serotype DJ or 2], and 100 μg of pZac-CASI-GFP or pZac-CMV-CasRx-gRNAs were mixed in 5 ml of DMEM, and 2 mg of PEI “MAX” (Polysciences) (40 kDa, 1 mg/ml in H₂O, pH 7.1) added for PEI:DNA mass ratio of 5:1. The mixture was incubated for 15 min, and transferred drop-wise to the cell media. The day after transfection, media was changed to DMEM+glutamax+pyruvate+2% FBS. Cells were harvested 48-72 hrs after transfection by scrapping or dissociation with 1×PBS (pH7.2)+5 mM EDTA, and pelleted at 1500 g for 12 min. Cell pellets were resuspended in 1-5 ml of lysis buffer (Tris HCl pH 7.5+2 mM MgCl+150 mM NaCl), and freeze-thawed 3× between dry-ice-ethanol bath and 37° C. water bath. Cell debris was clarified via 4000 g for 5 min, and the supernatant collected. The collected supernatant was treated with 50 U/ml of Benzonase (Sigma-Aldrich) and 1 U/ml of RNase cocktail (Invitrogen) for 30 min at 37° C. to remove unpackaged nucleic acids. After incubation, the lysate was loaded on top of a discontinuous density gradient consisting of 6 ml each of 15%, 25%, 40%, 60% Optiprep (Sigma-Aldrich) in an 29.9 ml Optiseal polypropylene tube (Beckman-Coulter). The tubes were ultra-centrifuged at 54000 rpm, at 18° C. for 1.5 hr, on a Type 70 Ti rotor. The 40% fraction was extracted, and dialyzed with 1×PBS (pH 7.2) supplemented with 35 mM NaCl, using Amicon Ultra-15 (100 KDa MWCO) (Millipore). The titer of the purified AAV vector stocks were determined using real-time qPCR with ITR-sequence-specific primers and probe, referenced against the ATCC reference standard material 8 (ATCC). EV-A71 lab strain is from strain 5865/sin/000009, GenBank accession no. AF316321.

### Immunofluorescence Assay

10,000 human immortalized corneal endothelial cells, B4G12, were plated on glass slides in a 48 well plate and transduced with AAVDJ-CasRx or AAVDJ-GFP at MOI 100K. At 3 days post-transduction, cells were fixed and permeabilized using methanol for 10 mins and blocked using 1×PBS with 5% BSA for 1 h. This is followed by primary antibody anti-HA (abcam) incubation at 1:200 dilution for 2 h at RT or anti-GFP (abcam) at 1:1500 for 2 h at room temperature. Secondary antibodies staining is carried out at 1:1000 (Thermo Fisher) for 2 h at room temperature. Slides were then washed 3× with 1×PBS and mounted on to the slide using ProLong mounting medium (Thermo). Images are taken using an Olympus microscope, exposure set at 15 ms and gain set at 6400 for bright field and the 488 channel, exposure is set at 800 us and gain set at 6400 for the DAPI channel.

### Bio-Panning Assay for Selection of AAV Serotype

Immortalized human RD cells or mouse C2C12 cells were seeded in a 48-well plate at a density of 10,000 cells per well in 200 μl DMEM containing 10% FBS. The cells were cultured overnight at 37° C. and allowed to adhere to the well. A panel of AAV (1,2,6,7,8,9, rh10, DJ and Anc80) were used to transduce the cells at MOI of 100K or 10K respectively, in triplicates. At 72 h post-transduction, the cells were harvested and the total GFP protein is quantitated using a GFP quantification kit (Biovision) on a multi-well plate reader (Tecan).

### RNA Gene Knockdown Activity in AAVDJ-CasRx Transduced Cells

Immortalized human RD cells were seeded in a 48-well plate at a density of 10,000 cells per well in 200 μl DMEM containing 10% FBS. The cells were transduced with AAV2-GFP at MOI of 10K for expression of GFP. The GFP knockdown efficiency of guide1 only, guide2 only and guide1+guide2 are tested by transduction of the GFP-expressing RD cells with AAV2-CasRx bearing the respective guides at of MOI 100K and MOI 1000K. At 72 h post-transduction, the cells were harvested and the total GFP protein is quantitated using a GFP quantification kit (Biovision) on a multi-well plate reader (Tecan).

In Vitro Antiviral Plaque Assay with EV-A71

For screening of anti-EV-A71 activity, RD cells were seeded in 96-well plates at a density of 10⁴ cells per well and incubated overnight in an incubator. Perform dilution of AAV2-CasRx bearing the different guides for transduction individually or in pooled format at MOI IK, 10K, 100K and 1000K in a 100 μl volume. At 72 h post-transduction, the cells were infected with EV-A71 virus at MOI of 1. The plate was washed twice with 1×PBS and incubated with DMEM with 2% FBS for 12 h. The supernatant from each well were harvested at 12 h post-infection and used for subsequent virus plaque assay. For virus plaque assay, RD cells were seeded in 24-well plates and incubated with 10 to 106 fold serially diluted supernatant samples in a 100 μl volume. Plates were washed twice with PBS and overlay media were added to each well and incubated with 15 mins rocking interval for an hour before leaving it in the incubator for 4 days. After 4 days of incubation, the overlay media was removed and crystal violet stain was added to visualize the plaques for counting and the infectious virus titre was calculated, expressed as the average number of PFU per millilitre (PFU/ml) of sample.

### RNA-Seq Library Preparation, Sequencing, and Analysis

RD cells were transduced with AAV2-CasRx-EV71pool at an MOI of 1000K at 8 hours post-seeding. Total RNA was extracted from RD cells 6 days later using the RNeasy Plus Mini kit from QIAGEN. Stranded mRNA libraries were prepared using the NEBNext II Ultra Directional RNA Library Prep Kit from New England Biolabs (Cat #E7760S) and sequenced on an Illumina HiSeq 4000 with 150 nt paired-end reads. ˜25M total reads were de-multiplexed per condition. Sequenced reads were quality tested using FASTQC and mapped to the hg19 human genome using the 2.7.10a STAR aligner. Read alignment was performed using the default parameters. The genome index was constructed using the gene annotation provided with hg19 Illumina iGenomes collection and sjdbOverhang value of 100. Quantifications were performed using was performed with DESeq2 v1.34.0 using triplicates to compute within-group dispersion and contrasts to compare treated (EV-A71 gRNAs) and untreated conditions. Significant differentially expressed genes were defined as having a false discovery rate of FDR<0.01 and a log 2 fold change of >0.75. Volcano plots were generated with Enhanced Volcano v1.12.0 in R 4.1.2.

### Flow Cytometry

For titration of AAV2-GFP transduction, 10,000 cells were seeded on a 48-well plate and transduced with AAV2-GFP at MOI of IK, 10K, 100K, and 1000K or left untransduced as control. After 3 days, cells were harvested and suspended in flow cytometry buffer (0.5% BSA, 2 mM EDTA in 1×PBS) and passed through a 70 μm cell strainer before flow cytometry analysis by MACS Flow Cytometer (Miltenyi Biotec). To assess GFP knockdown by CasRx, 10,000 cells were seeded on a 48-well plate and transduced with AAV2-GFP at MOI of 10K, followed by AAV2-CasRx-GFP_gRNA1, AAV2-CasRx-GFP_gRNA2, or AAV2-CasRx-GFP_gRNA1+2 at MOI 100K or 1000K or without AAV2-CasRx. After 3 days, cells were harvested and suspended in flow cytometry buffer (0.5% BSA, 2 mM EDTA in 1×PBS) and passed through a 70 μm cell strainer before flow cytometry analysis by MACS Flow Cytometer (Miltenyi Biotec). To detect CasRx-HA expression, 100,000 RD cells were seeded per well on a 12-well plate before they were transduced with AAV2-CasRx-HA at an MOI of 1000K. After 3 days, cells were harvested and rinsed once with 1×PBS before fixing with Fixation Buffer (BioLegend #420801) at room temperature for 20 minutes. Cells were then rinsed thrice with 1× Intracellular Staining Permeabilization Wash Buffer (BioLegend #421002) before being stained with anti-HA primary antibody (abcam ab9110, 1:50) on ice for 30 minutes. Cells were rinsed thrice and then stained with Alexa Fluor™ 488-conjugated secondary antibody (Invitrogen #A-21206, 1:200) on ice, in the dark, for 30 minutes. After 3 more rinses, cells were suspended in flow cytometry buffer (0.5% BSA, 2 mM EDTA in 1× analysis by MACS Flow Cytometer (Miltenyi Biotec). All flow cytometry results were analyzed using the FlowJo software.

qPCR for Human Innate and Adaptive Immune Response Genes Profiling

In a 6-well dish, 1 million immortalized human skeletal RD cells were seeded and transduced with AAV2-CRISPR-CasRx at MOI of 100K and 1000K. RNA of the transduced cells were harvested at 72 hrs post-transduction using the RNeasy universal plus mini kit (Qiagen). cDNA was synthesized using Superscript III (Thermo Fisher) and the immune response gene panel was quantitated using the RT2 profiling kit (Qiagen).

### Mouse Infections and Treatments

For toxicity analysis, BALB/c mice were injected intraperitoneally with either a dose of saline or 1×10¹² viral genomes (vgs) of AAVDJ GFPgRNA2 or 1×10¹¹ vgs or 1×10¹² vgs of AAVDJ-EV71gRNAs per mouse at 2 days old. For prophylaxis, BALB/c mice were injected intraperitoneally with 1×10¹¹ viral genomes (vgs) or 1×10¹² vgs of AAVDJ-EV71gRNAs per mouse at 2 days old and subsequently injected intraperitoneally with EV-A71 at a dose of 2×10⁷ PFU per mouse at 5 days old. For therapeutic analysis, BALB/c mice were injected intraperitoneally with a dose of 2×10⁷ PFU EV-A71 per mouse at 5 days old. After 6 hours or 24 hours, mice were injected intraperitoneally with a dose of 1×10¹¹ viral genomes (vgs) or 1×10¹² vgs of AAVDJ EV71gRNAs per mouse. 1×10¹² vgs AAVDJ-GFPgRNA2 was used as control. The survival of the mice and the clinical scores of each mouse in each treatment group was recorded daily for up to 19 days post-infection (dpi). Mice were scored in 5 categories for symptoms observed during the 19-day survival study. The scoring system includes Activity: 0 normal, 1 lethargy/abnormal posture, 2 huddled/inactive, 3 Unresponsive to stimuli, severe tremor and inability to right itself: Breathing: 0 normal, 1 rapid/shallow, 2 rapid abdominal, 3 labored, blue: Movement: 0 normal, 1 weakness, incoordination, 2 single limb dragging/paralysis, 3 multiple limb dragging/paralysis: Body weight: 0 normal, 1 loss of 5% over 24 h, 2 loss of more than 15% or up to 10% in 24 h, 3 loss of more than 10% over 24 h or 20% in total, body condition 2 or below: Dehydration: 0 normal skin tent, 1 skin tent present on dorsum, 2 Moderate skin tent, 3 Severe skin tent. A total of 6 or more points accumulated across all categories was determined as a humane endpoint, and mice with such a score were euthanized. Histological analyses were carried out on mice sacrificed at 6 days post-infection.

### Histology of Mouse Tissues

Histological samples were fixed in 4% paraformaldehyde at 4° C., decalcified at room temperature for 2 h, then embedded in paraffin and processed into 4 μm sections. Tissue pathology was evaluated by hematoxylin and eosin (H&E) staining and EVA71 antigen was detected by immunohistochemistry staining (IHC) using commercially available anti-EV-A71 antibody (MAB979, 1:500 dilution) and image captured using the Leica Bond-Max system. For staining of CasRx, histological slides were baked at 50° C. for 10 min and de-waxed in Xylene 3× for 5 min. Slides were then rehydrated in descending grade ethanol: 100% ethanol 2× for 5 min, 95% ethanol 5 min, 80% ethanol 3 min, 70% ethanol 3 min, 50% ethanol 3 min, rinsing with water, and rinsing with 1×PBS 3× for 5 min. Antigen retrieval was conducted by 2100-Retriever steam cooker (Prestige Medical) and slides were heated for 12-15 min in 0.01M Sodium Citrate buffer pH 6.0, cooled for 3-4 h, washed once in 1×PBS for 5 min, then washed using 1×PBS with 0.1% Triton 3× for 5 min each. Slides were blocked in 2% BSA+5% goat serum-PBS for 60 min at RT, then incubated with primary Ab 1:100 (anti-HA mouse, ab18181) in blocking buffer overnight at 4° C. After washing with 1×PBS with 0.1% Triton 3× for 5 min, slides were incubated with secondary Ab 1:500 (Alexa Fluor 488, A21202) in washing buffer for 2 h at RT, followed by washing with 603 0.1% Triton-1×PBS 3× for 5 min and counterstained with DAPI. Images were captured using a confocal microscope (Leica).

### Quantification of EV-A71 Titre in Mice Tissues

EV-A71-infected mice were sacrificed on 6 days post-infection (dpi) and mice tissue, including hind limb and brain, were collected into CK14 homogenizing tubes (Bertin Corp), homogenized and titrated by plaque forming assay. The tissues were weighed and 1 mL and 0.5 mL of DMEM for limb tissues and brain tissues was added, respectively into the tubes before homogenization using an orbital shaker at 6000×g for 10 s. The process was repeated 3 times for brain tissues and 9 times for limb tissues. The homogenized tissues were centrifuged for 10 min at 8000×g, 4° C. to pellet tissue debris. Supernatants were collected, and the viral load was titrated by viral plaque assay.

### Ethics Statement

Animal care and housing were provided in accordance with the National Advisory Committee for laboratory Animal Research (NACLAR) Guidelines (Guidelines on the Care and Use of Animals for Scientific Purposes). Experiments with mice were designed and approved under protocol 088/10 by National University of Singapore Institutional Animal Care & Use Committee (IACUC) based on NACLAR guidelines.

### Example 1

### Development of AAV-CRISPR-Cas Antiviral Modality for RNA Virus Targeting

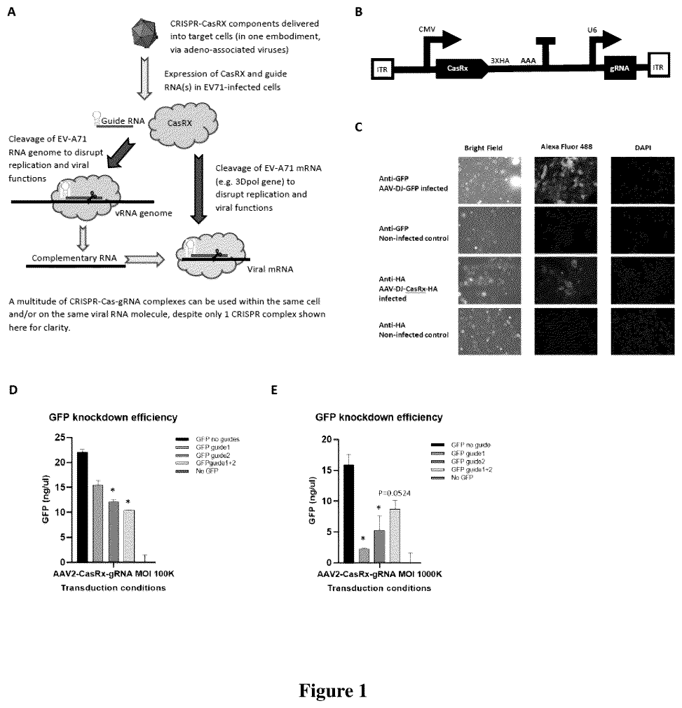

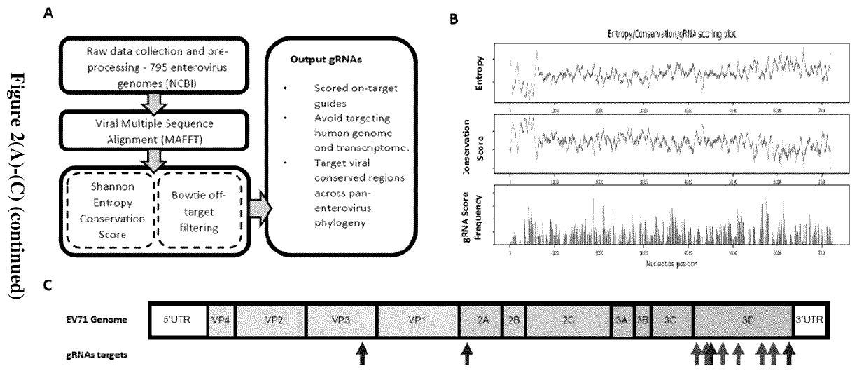

The EV-A71 life cycle is typical of a positive-strand single-stranded RNA virus, in which the virus enters the cell, releases its RNA genome into the cytoplasm, and synthesizes the negative-sense genomic and sub-genomic viral RNAs from which new copies of the positive sense viral genomes and transcripts are synthesized (FIG. 1A). In this study, it was proposed to use an AAV-vector based CRISPR-Cas13d system for direct targeting and cleavage of the viral intracellular viral genome and its resulting viral mRNAs to reduce viral replication (FIG. 1A). A new AAV cargo construct (FIG. 1B) was built, bearing the guide RNAs (gRNAs) and a CasRx that excludes the NLS sequences, so that the expressed CasRx protein has access to the viral replication complex that resides outside of the nuclear region. To validate the expression of the CasRx protein, immortalized human corneal endothelial cells were transduced with AAV-CRISPR-CasRx-gRNA and confirmed the expression and localisation of the HA-tagged CasRx protein in the cytoplasmic region via immunofluorescence (FIG. 1C). To test the efficacy of the new construct, AAV-CRISPR-CasRx-gRNAs that target the GFP mRNA sequence were generated and the viruses were tested on GFP-expressing cells (see Table 3 for gRNA sequences). The results confirmed that the NLS-free CasRx-gRNAs are functional and reduce GFP expression by up to 2.2-fold (MOI=100K) and up to 7-fold (MOI=1000K) (FIGS. 1D and 1E). GFP gRNA2 is more efficient for the GFP gene expression knockdown and hence was selected to be used as control gRNA in subsequent experiments.

### Computational Guide RNAs Design and Testing of Strategies for CRISPR-Cas Targeting of RNA Viruses

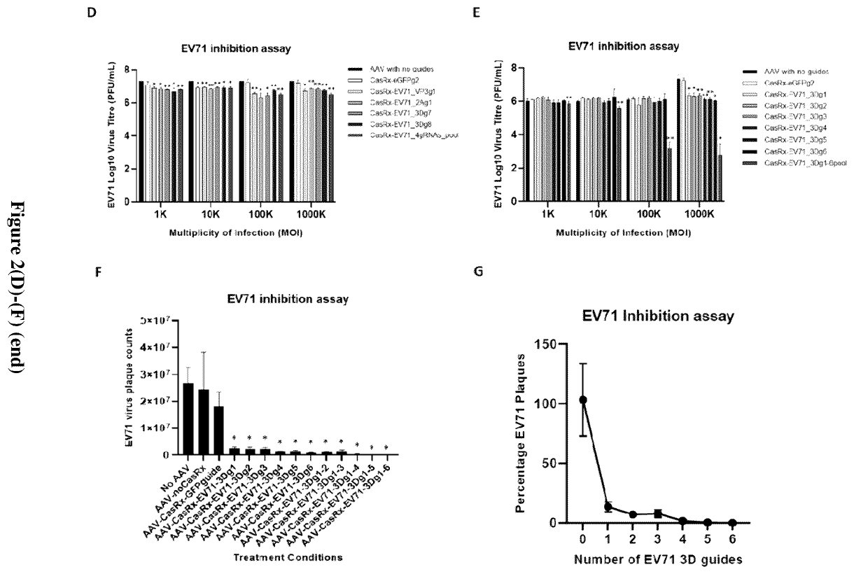

To aid in the design and selection of antiviral CRISPR-gRNAs, Cas13gRNAtor was developed. Cas13gRNAtor is a bioinformatics tool that generates all possible gRNA candidates for a user-provided genetic or genomic sequence, then scores each gRNAs via multiple parameters: (i) multiple sequence alignment of target genes/genomes to defined conserved regions with entropy and conservation scores, (ii) structural information (RNA folding, hybridization energy), (iii) on-target scores for predicted efficacy, and (iv) off-target scores for specificity (FIGS. 2A and 2B). Cas13gRNAtor was applied to curate an in silico list of candidate gRNAs that target enteroviruses within the highly conserved viral regions (so as to anticipate and avoid mutational escape) and are highly dissimilar to and hence not expected to exhibit off-targeting of the human transcriptome. The top 4th quartile of gRNAs was filtered based on guide score, eliminated gRNAs predicted to have potential off-target sites within the human transcriptome (HG38: including non-coding RNA), and eliminated gRNAs with T-homopolymers (>3 Ts), thereby obtaining a subset of 1265 candidate gRNAs. Conservation score at each nucleotide position was calculated as the percentage of sequences matching the consensus at that nucleotide position after a multiple sequence alignment was performed. A score of 1.0 indicates that the nucleotide position is fully conserved across all sequences. Cas13gRNAtor further narrowed to 56 candidate gRNAs that target highly conserved viral sequences with a mean conservation score of at least 0.9 for both gRNA and the protospacer intolerant region.

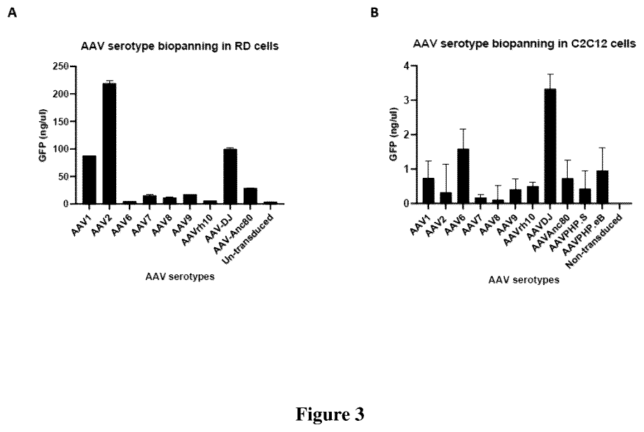

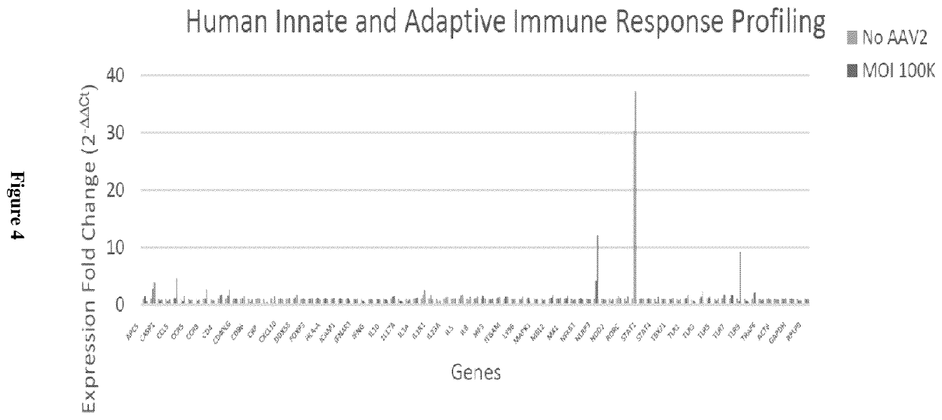

To efficiently deliver the CRISPR-Cas system into human skeletal RD cells that serve as the in vitro model for EV-A71 infection, an AAV serotype bio-panning experiment was performed on these cells to determine the AAV serotype that best transduces the cells. Among 9 serotypes, AAV2 was identified as the most efficient serotype for human skeletal RD cells (FIG. 3A).

It was then investigated if there are any differences in the activity of the individual guides based on their target gene or guide scores. AAV2-CRISPR-CasRx bearing each of the individual guides were used for transduction of RD cells at MOI of IK, 10K, 100K and 1000K followed by infection of the cells using EV-A71 at MOI of 1 at 72 hours post-transduction. The supernatant is then harvested at 12 h post infection for plaque assay. The results suggest that for individual high scoring guides, significant virus inhibition was observed at AAV-CRISPR-CasRx-gRNA at low MOIs of IK, 10K and 100K. For gRNAs targeting the VP3 and 3D gene, inhibition of up to 1 log reduction of virus titre (up to 90.8% of viral reduction) was observed (FIG. 2D). For low scoring gRNAs, they do not exhibit significant inhibition of virus replication at AAV-CRISPR-CasRx-gRNA MOI of IK, 10K or 100K, but all gRNAs exhibit significant inhibition of virus replication of up to 1 log reduction of virus titre at AAV-CRISPR-CasRx-gRNA MOI of 1000K (FIG. 2E). This demonstrate that Cas13gRNAtor can identify single gRNAs for efficient inhibition of viral replication. The result is also consistent with recent Cas13 antiviral studies that observed virus inhibition by better-performing individual guides can inhibit up to 90% of virus replication titres or reporter activities in vitro. To confirm that the viral inhibition is not due to any intracellular antiviral activity stimulated by the AAV delivery vector, a host human innate and adaptive immune response profiling was preformed using AAV2-CasRx-GFPgRNA2 transduced RD cells at MOI of 100K and 1000K. The result showed that there is minimal intracellular antiviral response in the AAV2-CasRx-GFPgRNA2 transduced RD cells with only two genes (NLRP3 and SLC11A1) being significantly upregulated in a dose-dependent manner (FIG. 4). NLRP3 and SLC11A1 are both involved in macrophage activation or recruitment but has not been shown to exhibit intracellular antiviral activity. With the minimal innate immunity induced intracellularly and the absence of viral plaque inhibition activity from the AAV2-CasRx-GFPgRNA2 negative controls, it was concluded that the viral inhibitory function of antiviral AAV-CasRx-gRNAs is specific and consequential of the gRNA-targeted CRISPR-CasRx endonuclease activities.

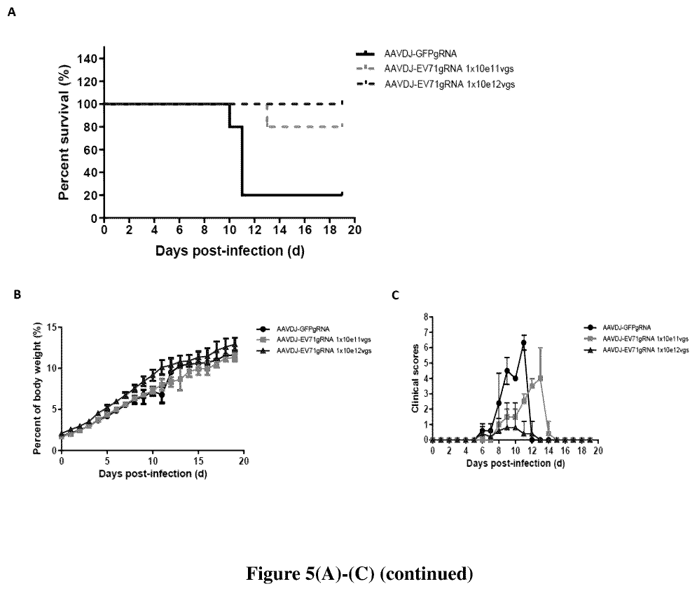

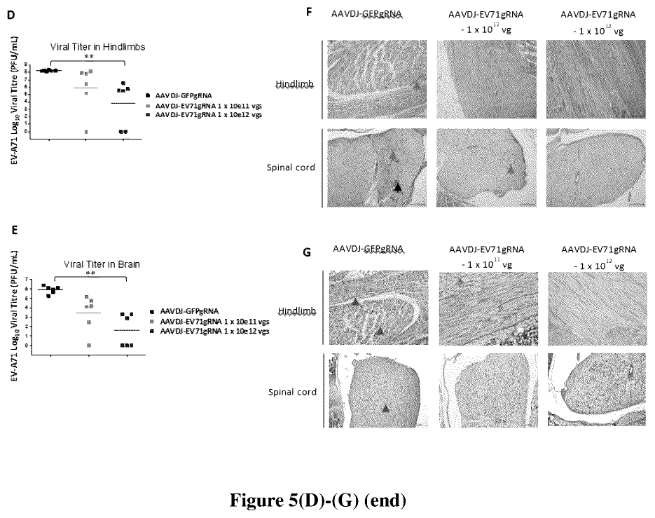

To develop a more potent AAV-CRISPR-Cas antiviral, different strategies of pooled gRNAs targeting were explored, namely multi-guides multi-genes targeting or multi-guides single-gene targeting approaches. 4 candidate gRNAs targeting different genes (2A, VP3 and 3Dpol) and 4 candidate gRNAs targeting only one gene (3Dpol) were shortlisted for comparison of antiviral efficacy. The EV-A71 3Dpol function as a RNA-dependent RNA polymerase (RdRP) and is an essential gene for the viral RNA synthesis in the replication cycle. RdRP genes are a common target for the development of viral inhibitors and antiviral therapeutics via blockade of viral replication. The entropies and conservation scoring of these individual guides is displayed in FIG. 5. The first pool of four candidate gRNAs targets the EV-A71 genomic and sub-genomic regions in 3D, VP3, and 2A with high in silico predicted scores while the second pool of four candidate gRNAs targets the EV-A71 genomic and sub-genomic regions in 3Dpol with low in silico predicted scores (see FIG. 2C and Table 1). In the multi-guides multi-genes approach, one or two gRNAs targeting each gene with target sites residing in the 2A, VP3 and 3D gene sequences was chosen. This initial gRNAs pooling approach exhibits significant inhibition of viral replication titre by up to 1 log of virus titre and up to 91.1% of viral reduction at MOI of 100K and 1000K, but is not significantly better than the single guides approach (FIG. 2D). In comparison with the multi-guides single-gene approach where 4 gRNAs were targeting to the same gene 3Dpol, a significant inhibition of up to 2 logs or up to 98.8% of virus titre reduction was observed (FIG. 2F). This result suggests that targeting all 4 gRNAs to the same 3D gene is more effective than having the 4 gRNAs targeting different genes on the viral sequence. Next, the differences in the inhibitory activities of the different number of pooled guides for the multi-guides single-gene approach were investigated by sequentially increasing the number of guides targeting the 3D gene. The results showed that increasing the number of pooled gRNAs for the same gene target up to three gRNAs did not significantly improve the inhibition which stayed at approximately 1 log of virus titre reduction while increasing the number of pooled gRNAs targeting the same gene target to four gRNAs significantly increases the inhibition up to 2 logs or up to 98.8% of virus titre reduction (FIGS. 2F and 2G). Further increasing the number of gRNAs targeting the same gene to five increases the inhibition up to 3 logs or up to 99.7% (FIGS. 2F and 2G). The most potent cocktail for the number of gRNAs tested in this study is six with observed inhibition of up to 5 logs or up to 99.99% of virus reduction at MOI of 100K or 1000K (FIGS. 2E, 2F and 2G). Even at a lower MOI of 10K, a significant inhibition of up to 1 log of virus titre or up to 90.1% of viral reduction is observed (FIG. 2E). Together, these data suggest that the multi-guides single-gene targeting strategy of up to six gRNAs can provide an effective method to eliminate EV-A71 RNA viruses in vitro.

### Effective Elimination of Viruses In Vivo in EV-A71-Infected Mouse Model

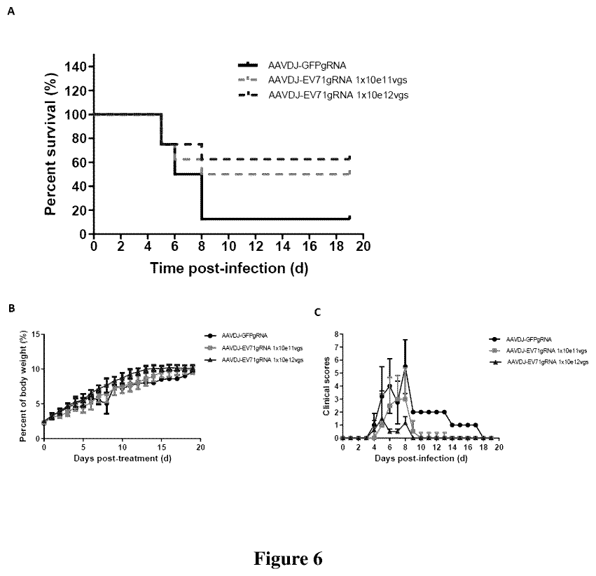

The effectiveness of the multi-guides single-gene approach was next investigated for the AAV-CRISPR-CasRx antiviral modality in an established EV-A71 murine model for hand, foot and mouse disease. To ensure efficient delivery of the therapeutics into the mouse muscle tissue which is a primary replication target for the EV-A71 virus, an AAV serotype bio-panning experiment was performed using the immortalized mouse C2C12 skeletal muscle cells, which identified AAVDJ serotype as the most efficient among the 11 serotypes assessed (FIG. 3B). To test the efficacy of the pooled AAVDJ-CRISPR-CasRx-gRNAs in the mouse model, 2-day old mice were first injected with either AAVDJ-CRISPR-CasRx-GFPgRNA2 at 1×10¹² total vector genomes (vgs) as the control treatment group or AAVDJ-CRISPR-CasRx-3D_gRNAs at 1×10¹¹ or 1×10¹² total vgs as the treatment group. After 3 days. the mice were then injected intraperitoneally with a lethal dose of 2×10⁷ PFU of EV-A71 per mouse and monitored daily for survivability, clinical symptoms, and body weight. As shown in FIG. 5A, 20% survival was observed at 11 days post-infection in the control group mice (n=5). In contrast, 80% survival was observed in the 1×10¹¹ vgs AAVDJ-CRISPR-CasRx treatment group (n=5) at 13 days post-infection and up to 19 days post-infection. The higher treatment dose of 1×10¹² vgs conferred 100% protective effect in the infected mice (n=7, logrank Mantel-Cox test. *p=0.0003) up to 19 days post lethal infection. Based on physical symptoms of body weight, activity, breathing, movement, and dehydration, higher clinical scores and lower body weight of EV-A71-infected mice were observed in the control treated group when compared with the two treatment groups which showed minimal clinical symptoms (FIGS. 6B and 6C). EV-A71-infected mice in the control treatment group developed severe clinical symptoms which include inactivity, loss of body weight, and hind limbs paralysis.

To examine the impact on viral infection and pathology that the AAV-CRISPR-CasRx antiviral modality has within the established EV-A71 murine model, mice muscle tissues and brains of the EV-A71-infected mice were harvested at 6 dpi and the viral titers were quantified by viral plaque assay. The results showed that while viral titer remain detectable in the hind limb and brain tissues upon treatment in a few mice, a large reduction in viral titer was observed in most treated mice. In hind limbs, viral titration showed about 2 to 3-log reduction in 4 mice and viral titer was not detected in 2 mice of the 1×10¹² total vgs treated mice group (p=0.0030, Kruskal-Wallis test) compared to control group (FIG. 5D). In brain tissues, viral titration showed 3-log reduction in 3 mice and viral titer was not detected in 3 mice of the 1×10¹² total vgs treated mice group (p=0.0017, Kruskal-Wallis test) compared to control group (FIG. 5E). Results from H&E staining performed on the limb muscle and spinal cord tissues revealed no obvious muscle necrosis in AAVDJ-CRISPR-CasRx-3D_gRNAs with 1×10¹² total vgs and mild necrosis was noted for 1×10¹¹ total vgs treated mice compared to severe necrosis observed in control group (FIG. 5F). No viral antigen distribution was observed in 1×10¹² total vgs treated mice, while minimal viral antigen was present in 1×10¹¹ total vgs treated mice and severe viral antigen distribution was observed in controls mice for both hind limbs and mainly the cervical spinal cord anterior horn neurons (FIG. 5G).

Importantly, the AAV-CRISPR-CasRx antiviral modality is effective as a therapeutic intervention in mice already infected with EV-A71. To test the efficacy of the pooled AAVDJ-CRISPR-CasRx-gRNAs in the mouse model, 5-day old mice were first injected intraperitoneally with a lethal dose of 2×10⁷ PFU of EV-A71 per mouse. Then, 2 hours later, the mice were injected intraperitoneally with AAVDJ-CRISPR-Cas-GFPgRNA2 at 1×10¹² vgs or AAVDJ-CRISPR-CasRx-3D_gRNAs at 1×10¹¹ or 1×10¹² total vgs (FIG. 6). The mice were monitored daily for survivability, clinical symptoms, and body weight. These results demonstrated that AAVDJ-CRISPR-CasRx-3D_gRNAs at 1×10¹¹ vgs successfully conferred survival of 50% of the mice (n=8) and 1×10¹² total vgs conferred survival of 62.5% (n=8) of the mice up to 19 days post-lethal infection. Based on physical symptoms, higher clinical scores and lower body weight of EV-A71-infected mice were observed in the control group compared with the 1×10¹¹ and mainly in 1×10¹² total vgs treatment group which showed minimal clinical symptoms and a greater increase in body weight.

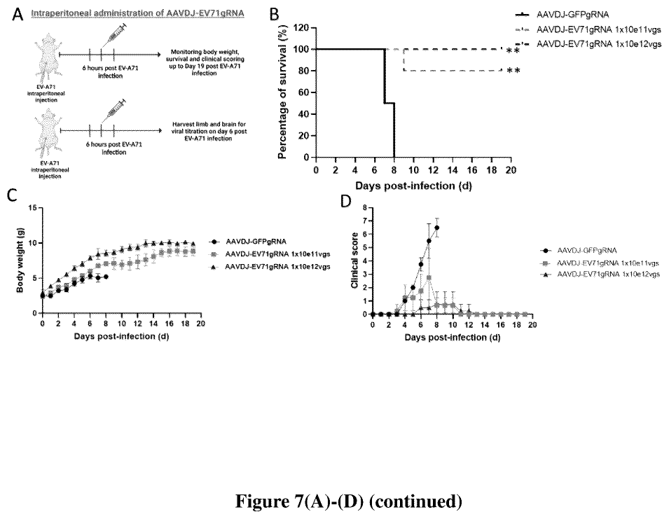

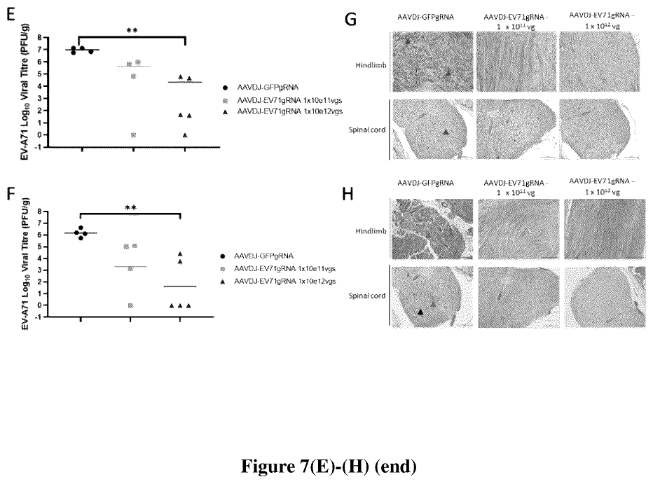

In another experiment, 5-day-old mice were injected intraperitoneally with a lethal dose of 2×10⁷ PFU of EV-A71 per mouse. After 6 hours, the mice were intraperitoneally injected with AAVDJ-CRISPR-Cas-GFPgRNA2 at 1×10¹² vgs or AAVDJ-CRISPR-CasRx-3D_gRNAs at 1×10¹¹ or 1×10¹² total vgs. The mice were observed daily for survivability, clinical symptoms, and body weight (FIG. 7A). The results indicate that AAVDJ-CRISPR-CasRx-3D_gRNAs at 1×10¹¹ vgs successfully conferred survival of 80% to the mice (n=5, logrank Mantel-Cox test: *p=0.0039) and 1×10¹² total vgs conferred survival to 100% of the mice (n=4, logrank Mantel-291 Cox test: *p=0.0084) up to 19 days post lethal infection (FIG. 7B). Lower clinical symptoms, better body weight, significantly reduced viral titers in hind limbs and brains, significantly reduced viral antigens in tissues, and alleviation of muscle necrosis were observed in the treatment groups (FIG. 7C to 7H).

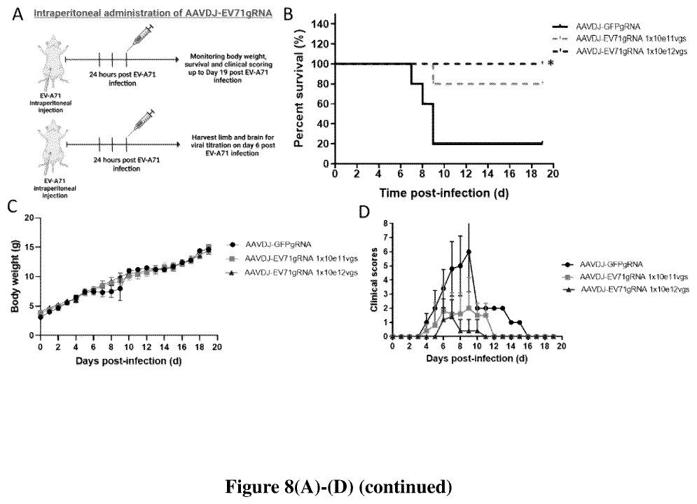

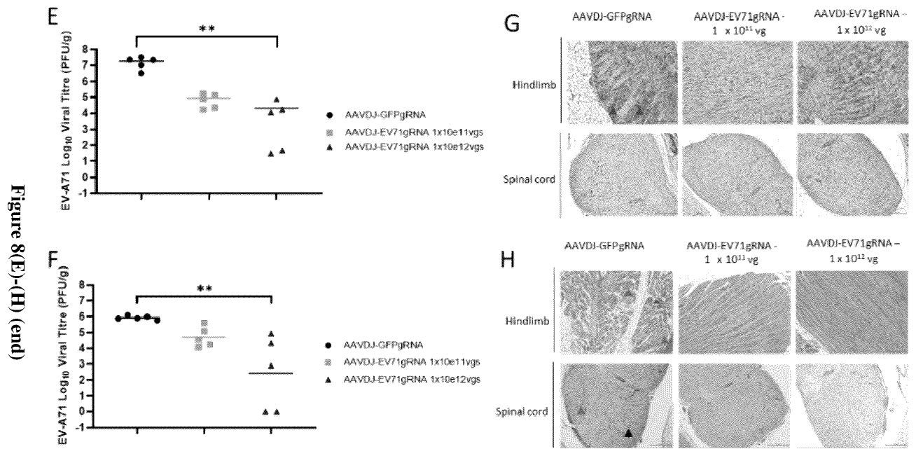

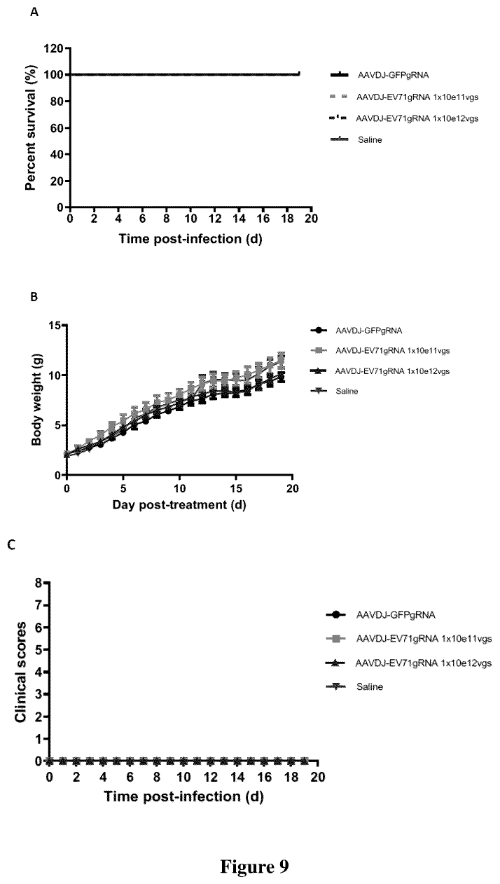

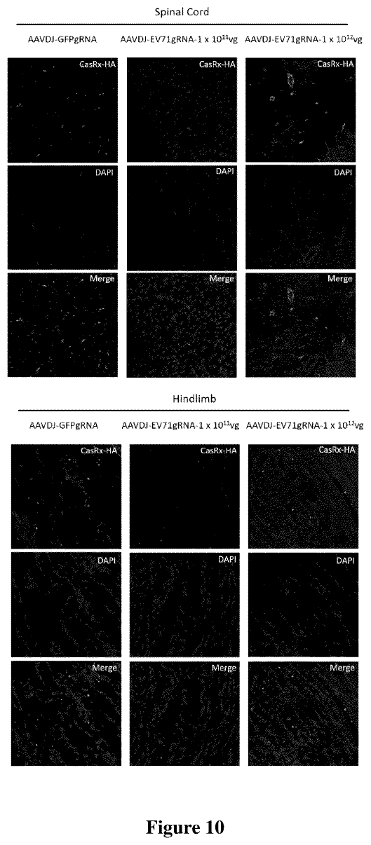

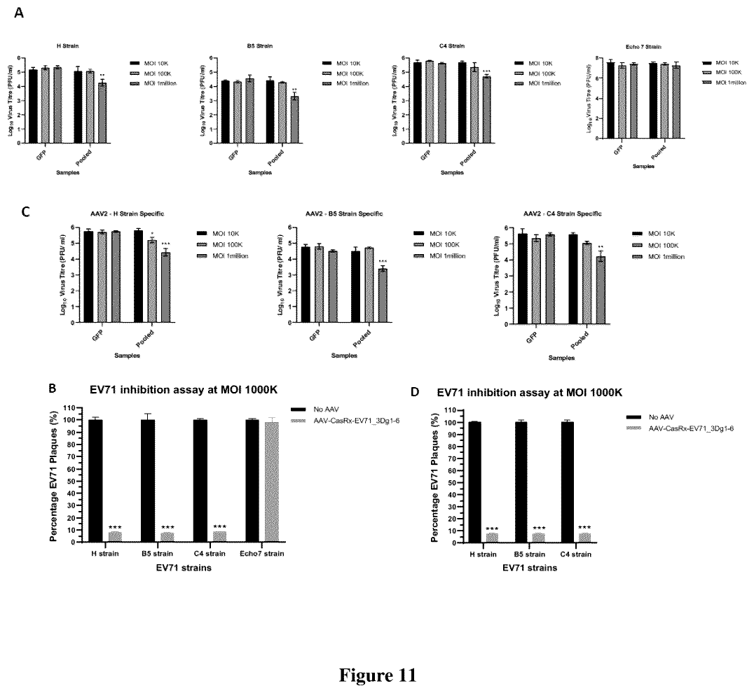

Importantly, even at an extended even at an extended 24 h post-infection treatment (FIG. 8A), AAVDJ CRISPR-CasRx-3D_gRNAs at 1×10¹¹ vgs successfully conferred survival to 80% of the mice (n=5, logrank Mantel-Cox test: *p=0.053) and at 1×10¹² total vgs conferred survival to 100% of the mice (n=5, logrank Mantel-Cox test: *p=0.015) up to 19 days post lethal infection (FIG. 8B). Treatment groups again exhibited improved clinical scores and body weights (FIGS. 8C and 8D). Viral titers were significantly reduced by 3 logs in the hind limbs (p=0.0018, Kruskal-Wallis test) (FIG. 8E) and 4 logs in the brain tissues (p=0.0029, Kruskal-Wallis test) (FIG. 8F) of mice in the 1×10¹² total vgs treatment group compared to the control group. IHC in hind limbs and cervical spinal cord anterior horn neurons showed minimal viral antigen distribution in mice treated with 1×10¹² total vgs, modest viral antigen in mice treated with 1×10¹¹ total vgs, and severe viral antigen distribution in control mice (FIG. 8G). H&E staining performed on the limb muscle and spinal cord tissues revealed that AAVDJ-CRISPR-CasRx-3D_gRNAs alleviates the severe muscle necrosis observed in the control group (FIG. 8H). Taken together, the results indicate that the AAVDJ-CRISPR-CasRx-3D_gRNAs modality is effective as a treatment against EV-A71 throughout the treatment window pre-infection and at 2 h, 6 h and 24 h post-infection. Importantly, the AAVDJ-CRISPR-CasRx-3D_gRNAs treatment prevents death and eliminates up to 99.9% of the EV-A71 RNA viruses in vivo.

TABLE 1
EV-A71 gRNAs sequences and scorings

| | | SEQ | | | |
|---|---|---|---|---|---|
| gRNA | | ID | | Entropy | Conservation |
| Name | DNA encoding gRNA spacer sequence | NO: | Guide Score | Score | Score |
| 3Dg1 | GTTGGTCCATTGATGTTTAGTCT | 1 | 0.011814542 | 0.22192 | 0.94165 |
| 3Dg2 | TTGGAAAACAGGGCTTGTTCAAA | 2 | 0.049176595 | 0.24443 | 0.92918 |
| 3Dg3 | TAGCACGCTTCCTCCATGCTCAT | 3 | 0.56835903 | 0.17175 | 0.95543 |
| 3Dg4 | TATTTATCCATGTAGAATTTCAT | 4 | −0.06258042 | 0.17843 | 0.94914 |
| 3Dg5 | ATGATGTTGTTGATCATTGAATT | 5 | 0.080602909 | 0.14546 | 0.96782 |
| 3Dg6 | TCTGCAGGAGTCATGGTCAAACC | 6 | 0.477791799 | 0.24641 | 0.93043 |
| 3Dg7 | CAAAGTTAGGAATGGCCAACGCT | 7 | 1.303861968 | 0.30996 | 0.9162 |
| 3Dg8 | CACATACTTGGAAAACAGGGCTT | 8 | 1.074780839 | 0.22671 | 0.94192 |
| 2Ag1 | AACCACTCTGAAGTTGCCCACGT | 9 | 1.241615481 | 0.19715 | 0.94526 |
| VP3g1 | GGTGACAGATGATTGTAGCCCAA | 10 | 0.673976462 | 0.19415 | 0.95089 |

TABLE 2
EV-A71 gRNAs sequences

| gRNA | | |
|---|---|---|
| Name | gRNA spacer sequence | SEQ ID NO: |
| 3Dg1 | GUUGGUCCAUUGAUGUUUAGUCU | 11 |
| 3Dg2 | UUGGAAAACAGGGCUUGUUCAAA | 12 |
| 3Dg3 | UAGCACGCUUCCUCCAUGCUCAU | 13 |
| 3Dg4 | UAUUUAUCCAUGUAGAAUUUCAU | 14 |
| 3Dg5 | AUGAUGUUGUUGAUCAUUGAAUU | 15 |
| 3Dg6 | UCUGCAGGAGUCAUGGUCAAACC | 16 |
| 3Dg7 | CAAAGUUAGGAAUGGCCAACGCU | 17 |
| 3Dg8 | CACAUACUUGGAAAACAGGGCUU | 18 |
| 2Agl | AACCACUCUGAAGUUGCCCACGU | 19 |
| VP3g1 | GGUGACAGAUGAUUGUAGCCCAA | 20 |

TABLE 3
GFP gRNA sequences

| gRNA | | SEQ ID |
|---|---|---|
| Name | Guide Sequence | NO: |
| GFPg1 | GTGAACAGCTCCTCGCCCTTGCT | 21 |
| GFPg2 | GCTGAACTTGTGGCCGTTTAC | 22 |

1. A method of preventing or treating an RNA viral infection in a subject, the method comprising administering at a dose of about 5×10¹¹ to about 5×10¹³ vgs/kg of a recombinant adeno-associated virus (AAV) to the subject, wherein the AAV comprises at least one heterologous nucleic acid sequence encoding a Cas13 nuclease and one or more guide RNAs.

2-15. (canceled)

16. The method of claim 1, wherein the dose is about 5×10¹² to about 5×10¹³ vgs/kg.

17. The method of claim 1, wherein the RNA virus infection is an infection by a single stranded RNA virus.

18. The method of claim 1, wherein the single stranded RNA virus is selected from the group consisting of an Enterovirus, a Coxsackie virus and a Parechovirus.

19. The method of claim 18, wherein i) the Enterovirus is Enterovirus 71; ii) the Coxsackie virus is selected from the group consisting of CAV16 and CAV6; and iii) the Parechovirus is selected from the group consisting of Parechovirus A, Parechovirus B, Parechovirus C, Parechovirus D, Parechovirus E, and Parechovirus F.

20. The method of claim 19, wherein the Cas13 nuclease is a Cas13a, Cas13b, Cas13c or Cas13d nuclease.

21. The method of claim 1, wherein the AAV vector is an AAV2, AAVDJ or AAV1 vector.

22. The method of claim 1, wherein the at least one guide RNA comprises i) a first nucleic acid sequence having at least 70% sequence identity to a nucleic acid sequence encoded by one of the nucleic acid sequences set forth in SEQ ID NO: 1-10, or ii) a first nucleic acid having at least 70% sequence identity to one of the nucleic acid sequences set forth in SEQ ID NO: 11-20.

23. The method of claim 1, wherein the Cas13 nuclease is operably linked to a CMV promoter.

24. The method of claim 1, wherein the guide RNA is operably linked to a U6 promoter.

25. The method of claim 1, wherein preventing or treating the RNA viral infection comprises inhibiting the RNA viral nucleic acid in a skeletal or central nervous system (CNS) cell or tissue of the subject.

26. A method of inhibiting an RNA viral nucleic acid in a subject, the method comprising administering about 5×10¹¹ to about 5×10¹³ vgs/kg of a recombinant adeno-associated virus (AAV) to the subject, wherein the AAV comprises at least one heterologous nucleic acid sequence encoding a Cas13 nuclease and one or more guide RNAs.

27. The method of claim 26, wherein the method comprises inhibiting the RNA viral nucleic acid in a skeletal or central nervous system (CNS) cell or tissue of the subject.
